(19) United States
(12) Patent Application Publication
(10) Pub. No.: US 2018/0277248 A1
(43) Pub. Date: Sep. 27, 2018

(54) TREATMENT RECOMMENDATIONS BASED ON BIOMARKER VALUES

(71) Applicant: Rejuvenan Global Health, Inc., New York, NY (US)

(72) Inventors: Fereydoun Fred Nazem, New York, NY (US); Joel Fuhrman, Flemington, NJ (US); Thomas B. Okarma, Palo Alto, CA (US)

(21) Appl. No.: 15/764,703

(22) PCT Filed: Sep. 30, 2016
(86) PCT No.: PCT/US16/54656

§ 371 Date: Mar. 29, 2018

Related U.S. Application Data
(60) Provisional application No. 62/234,984, filed on Sep. 30, 2015.

(51) Int. Cl. G16H 50/20; G16H 50/30

(52) U.S. Cl. CPC G16H 50/20; G16H 50/30

(57) ABSTRACT

A system, a method, and a non-transitory computer program product for generating treatment recommendations based on biomarker values are disclosed. Values of a plurality of biomarkers are received from an application executed on a computing device of a user. A score is generated for each biomarker. An overall heath score is computed for a user by weighting and adding all biomarker scores. A severity associated with each biomarker is computed. A physiological condition associated with the severity of each biomarker is identified. An explanation of the severity of the biomarker and at least one treatment recommendation are generated based on the severity associated with each biomarker. The score for each biomarker, the overall health score, the explanation of the severity, and the at least one treatment recommendation are transmitted to the application executed on the computing device of the user via a communication network.

### RELATED APPLICATION

This patent application claims priority to U.S. Provisional Patent Application Ser. No. 62/234,984 entitled “Systems and Methods for Treatment and/or Prevention of Diseases and/or their Complications”, and filed on Sep. 30, 2015, the content of which is incorporated herein by reference in its entirety.

### TECHNICAL FIELD

The subject matter described herein generally relates to data processing, and in particular to generating treatment recommendations based on biomarker values of a user.

### BACKGROUND

Many individuals in the country are at a risk of developing one or more diseases, such as obesity, hypertension, diabetes, cardiovascular diseases, poor nutrition, and so on. These individuals are also often prone to other complications, such as atherosclerosis, kidney disease, retinopathy, peripheral neuropathy, heart failure, chronic obstructive pulmonary disease, liver disease, and the like. A substantial set of individuals within this population fails to receive preventive care and/or care that addressing an existing condition, and ends up in the often uncomfortable or unsuccessful path of curing these diseases. Some conventional technologies provide generic educational guidance to the public, but do not provide customized guidance to individuals. Moreover, the traditional implementations are not interactive with users, but if they are then those interactions are not user-friendly, provide insufficient information without scientific justification, and are not prompt. Therefore, there exists a need to have a system and/or platform that can provide useful and customized information to a user in a timely manner and based on validated scientific justifications. The subject matter described herein addresses this need and provides additional benefits as well.

### SUMMARY

Some implementations of the current subject matter generally relate to a computing system that can: receive values of a plurality of biomarkers from an application executed on a computing device of a user, generate a score for each biomarker, compute an overall heath score for a user by weighting and adding all biomarker scores, compute a severity associated with each biomarker, identify a physiological condition associated with the severity of each biomarker, generate an explanation of the severity of the biomarker and at least one treatment recommendation based on the severity associated with each biomarker, and send the following to the application executed on the computing device of the user via a communication network: the score for each biomarker, the overall health score, the explanation of the severity, and the at least one treatment recommendation. Related methods, techniques, apparatuses, cloud computing systems, and non-transitory computer programmable products are also described.

In one aspect, one or more processors can receive values of a plurality of biomarkers from an application executed on a computing device of a user. The one or more processors can generate a plurality of biomarker scores for the plurality of biomarkers. The one or more processors can compute a severity associated with each biomarker of the plurality of biomarkers. The severity for the biomarker can be computed based on a biomarker score of the plurality of the biomarker scores that is associated with the biomarker. The one or more processors can identify a physiological condition and at least one treatment recommendation that are specific to the severity of each biomarker. The one or more processors can retrieve, from a database operably coupled to the one or more processors, one or more links to an explanation of the physiological condition of each biomarker and at least one treatment recommendation for each biomarker. The one or more processors can send the plurality of biomarker scores and the one or more links to the explanation of the severity and the at least one treatment recommendation to the application executed on the computing device of the user via a communication network.

In some variations, one or more of the following can be implemented either individually or in any feasible or suitable combination. The one or more processors can compute an overall heath score for a user by weighting and adding each biomarker score of the plurality of biomarker scores. The one or more processors can send the overall health score to the application executed on the computing device of the user via a communication network. Each biomarker can be associated with a predetermined weight. The predetermined weight can be used for weighting a biomarker score associated with the biomarker. The application simultaneously can display the plurality of biomarker scores, the one or more links to the explanation of the severity and the at least one treatment recommendation, and the overall health score on a single graphical user interface.

The severity for each biomarker can be one of: normal, mild, moderate and severe. The one or more processors can be arranged in a model view controller (MVC) architecture. The generating of the plurality of biomarker scores for the plurality of biomarkers can include: identifying a range within a plurality of ranges within which each value of the plurality of values lies; and determining, using a table stored in the database, a biomarker score of the plurality of biomarker scores that is associated with the determined range. The retrieving of a link of the explanation of the physiological condition of each biomarker can include identifying the explanation amongst a plurality of explanations for which separate links are stored in the database. The plurality of explanations can include a separate explanation for each severity of each biomarker.

The retrieving of a link of the at least one treatment recommendation for each biomarker can include identifying the at least one treatment recommendation amongst a plurality of treatment recommendations for which separate links are stored in the database. The plurality of treatment recommendations can include a separate treatment recommendation for each severity of each biomarker.

In another aspect, a non-transitory computer program product is described that can store instructions that, when executed by at least one programmable processor, cause the at least one programmable processor to perform the following operations. The at least one programmable processor can receive values of a plurality of biomarkers from an application executed on a computing device of a user. The at least one programmable processor can generate a plurality of biomarker scores for the plurality of biomarkers. The at least one programmable processor can compute an overall heath score for a user by weighting and adding each biomarker score of the plurality of biomarker scores. The at least one programmable processor can send the overall health score to the application executed on the computing device of the user via a communication network.

In some variations of the above-noted aspect, one or more of the following can be implemented either individually or in any feasible or suitable combination. The at least one programmable processor can compute a severity associated with each biomarker of the plurality of biomarkers. The severity for the biomarker can be computed based on a biomarker score of the plurality of the biomarker scores that is associated with the biomarker. The at least one programmable processor can identify a physiological condition and at least one treatment recommendation that are specific to the severity of each biomarker. The at least one programmable processor can retrieve, from a database operably coupled to the one or more processors, one or more links to an explanation of the physiological condition of each biomarker and at least one treatment recommendation for each biomarker. The at least one programmable processor can send the plurality of biomarker scores and the one or more links to the explanation of the severity and the at least one treatment recommendation to the application executed on the computing device of the user via a communication network.

The application can simultaneously display the plurality of biomarker scores, the one or more links to the explanation of the severity and the at least one treatment recommendation, and the overall health score on a single graphical user interface.

In yet another aspect, a system is described that can include a frontend component and a backend component, both of (for example, arranged according to) a model view controller (MVC) architecture. The frontend component can include an interaction module and a display module. The interaction module can receive a plurality of values of a plurality of biomarkers from a computing device. The backend component can include an assessment module and a recommendation module. The assessment module can generate a plurality of biomarker scores for the plurality of biomarkers based on the values of the biomarkers. The assessment module can determine a severity associated with each biomarker based on a biomarker score for the biomarker. The recommendation module can determine a treatment recommendation specific to each biomarker based on the severity for the biomarker. The assessment module can send the plurality of biomarker scores to the display module. The recommendation module can send each treatment recommendation to the display module.

In some variations of the aspect mentioned above, one or more of the following can be implemented either individually or in any feasible or suitable combination. The computing device can execute an application that can receive one or more values of the plurality of values from the computing device. The interaction module can receive the one or more values from the application. The display module can enable the display of the plurality of biomarker scores and the treatment recommendation specific to each biomarker on the computing device. The frontend component can further include a login module configured to receive authentication data of a user from the computing device. The backend component can further include a user module configured to receive the authentication data from the login module to authenticate the user. The assessment module can generate the plurality of biomarker scores after the authentication of the user.

The system can further include a database that can store a plurality of treatment recommendations that include a treatment recommendation specific to each severity level of each biomarker. The backend component can further include a persistence module. The persistence module can be operably coupled to the database. The plurality of treatment recommendations stored in the database can be made accessible via the persistence module.

Computer program products are also described that include non-transitory computer readable media storing instructions, which when executed by at least one data processors of one or more computing systems, causes at least one data processor to perform operations herein. Similarly, computer systems are also described that may include one or more data processors and a memory coupled to the one or more data processors. The memory may temporarily or permanently store instructions that cause at least one processor to perform one or more of the operations described herein. In addition, methods can be implemented by one or more data processors either within a single computing system or distributed among two or more computing systems.

The subject matter described herein provides many technical advantages. For example, the at least one treatment recommendation described herein can: 1) prevent or reduce disease progression within the user and the development of disease complications within the user, 2) reverse the disease or its complications within the user, and 3) reduce the need for medications the user is already taking for his/her physiological condition. The computing platform with an intuitive user-interface, personalized wellness contents and services can be easily accessed and implemented by the patient without the need for a healthcare provider. The present subject matter can be readily scaled to provide organizations and their employees tools to increase the overall health of the organizations as well as the individual employees. The current subject matter can allow an automation of a doctor's visit, thereby giving user more control of his/her life. The at least one treatment recommendations can be made continuously available on the computing device of the user for twenty four hours a day, seven days a week, and every day of the year.

The implementations described herein are advantageous over traditional medical interventions. For example, the at least one treatment recommendation described herein can include behavioral and/or lifestyle changes that the user can adopt early in the course of the development of a physiological disease or condition, such as when signs and symptoms of the user's condition may be mild or even non-existent, or when the symptoms of a disease are present but not yet severe enough to warrant pharmaceutical intervention. These behavioral and/or lifestyle changes, if adopted by the user, can decrease or improve the severity of symptoms of the physiological condition and/or prevent the condition from progressing to a more severe state. Contrarily, the traditional medical interventions or traditional medicine does not allow for an early enough therapeutic intervention for a certain physiological diseases or conditions compared to that normally used in traditional medicine.

The details of one or more variations of the subject matter described herein are set forth in the accompanying drawings and the description below. Other features and advantages of the subject matter described herein will be apparent from the description, the drawings, and the claims.

### DESCRIPTION OF DRAWINGS

FIG. 1 is a system diagram illustrating a computer-architecture of a system generating an overall health score of a user and treatment recommendations for the user based on values of biomarkers specific to that individual, according to some implementations of the current subject matter;

FIG. 2 is a flow diagram illustrating an exemplary process for generating an overall health score for the user, and generation of treatment recommendations for the user, according to some implementations of the current subject matter;

FIG. 3 is a flow diagram illustrating an exemplary process for providing treatment recommendations using the recommendation module, according to some implementations of the current subject matter;

FIG. 4 is a flow diagram illustrating a process for using the persistence module to provide at least one of biomarker score for each biomarker of user, overall health score for user, severity associated with each biomarker, and treatment recommendations for user to display module upon user demand or in real-time, according to some implementations of the current subject matter;

FIG. 5 is a flow diagram illustrating an exemplary process for using the persistence module to send data for display by the application executed on the computing device, according to some implementations of the current subject matter;

FIG. 6 illustrates a graphical user interface executed by the application when a user opens the application, according to some implementations of the current subject matter;

FIG. 7 illustrates an exemplary graphical user interface executed by the application to receive a value of the weekly activity level biomarker of the user, according to some implementations of the current subject matter;

FIG. 8 illustrates an exemplary graphical user interface executed by the application to receive a value of the alcoholic drinks consumed per week biomarker of the user, according to some implementations of the current subject matter;

FIG. 9 illustrates an exemplary graphical user interface executed by the application to receive a value of the A1C biomarker of the user, according to some implementations of the current subject matter;

FIG. 10 illustrates an exemplary graphical user interface executed by the application to receive a value of the LDL cholesterol biomarker of the user, according to some implementations of the current subject matter;

FIG. 11 illustrates an exemplary graphical user interface executed by the application to receive values of the waist, height, and weight biomarkers of the user, according to some implementations of the current subject matter;

FIG. 12 illustrates the graphical user interface of FIG. 11 where graphical features have been used by the user to input values of the waist, height, and weight biomarkers of the user, according to some implementations of the current subject matter;

FIG. 13 illustrates an exemplary graphical feature displayed by the application when the graphical feature for inputting the waist biomarker is selected in the graphical user interface of FIG. 11 or 12, according to some implementations of the current subject matter;

FIG. 14 illustrates an exemplary graphical feature displayed by the application when the graphical feature for inputting the height biomarker is selected by the user in the graphical user interface of FIG. 11 or 12, according to some implementations of the current subject matter;

FIG. 15 illustrates an exemplary graphical user interface executed by the application to receive values of the weekly nutrient intake biomarker of the user, according to some implementations of the current subject matter;

FIG. 16 illustrates an exemplary graphical user interface executed by the application to receive values of the weekly glycemic food intake biomarker of the user, according to some implementations of the current subject matter;

FIG. 17 illustrates an exemplary graphical user interface executed by the application to receive values of the average number of cigarettes smoked per day biomarker of the user, according to some implementations of the current subject matter;

FIG. 18 illustrates an exemplary graphical user interface executed by the application to receive values of the blood pressure biomarker of the user, according to some implementations of the current subject matter;

FIG. 19 illustrates an exemplary graphical user interface executed by the application to receive values of the vitamin D biomarker of the user, according to some implementations of the current subject matter;

FIG. 20 illustrates an exemplary graphical user interface executed by the application to display the biomarker score for each biomarker, a severity determined for each biomarker, a button which when selected by the user makes the application display data characterizing an explanation of the severity for specific biomarkers, and an overall health score when values of a threshold number of biomarkers has been received by the application, according to some implementations of the current subject matter;

FIG. 21 illustrates an exemplary graphical user interface executed by the application to display: data characterizing an explanation of the severity for the displayed biomarker, and a button which when selected by the user makes the application display data including a treatment recommendation based on the severity of that particular biomarker, according to some implementations of the current subject matter;

FIG. 22 illustrates an exemplary graphical user interface executed by the application to display: data characterizing respective explanations of the severities for the displayed biomarkers, and a button which when selected by the user makes the application display data including a treatment recommendation based on the severity of the particular biomarker corresponding to the selected button, according to some implementations of the current subject matter;

FIG. 23 illustrates an exemplary graphical user interface executed by the application to display: data characterizing respective explanations of the severities for the displayed biomarkers, and a button which when selected by the user makes the application display data including a treatment recommendation based on the severity of the particular biomarker corresponding to the selected button, according to some implementations of the current subject matter;

FIG. 24 illustrates an exemplary graphical user interface executed by the application to display: data characterizing respective explanations of the severities for the displayed biomarkers, and a button which when selected by the user makes the application display data including a treatment recommendation based on the severity of the particular biomarker corresponding to the selected button, according to some implementations of the current subject matter;

FIG. 25 illustrates an exemplary graphical user interface executed by the application to display: data characterizing respective explanations of the severities for the displayed biomarkers, and a button which when selected by the user makes the application display data including a treatment recommendation based on the severity of the particular biomarker corresponding to the selected button, according to some implementations of the current subject matter;

FIG. 26 illustrates an exemplary graphical user interface executed by the application to display: data characterizing an explanation of the severity for the displayed biomarker, and a button which when selected by the user makes the application display data including a treatment recommendation based on the severity of that particular biomarker, according to some implementations of the current subject matter;

FIG. 27 illustrates an exemplary graphical user interface executed by the application to display a link to data characterizing an explanation of a physiological condition when the user has the severity determined by the assessment module, and another link to data of at least one treatment recommendation for the user, according to some implementations of the current subject matter;

FIG. 28 illustrates an exemplary graphical user interface executed by the application to display a challenge program, which is one of multiple challenge programs that a user can select, according to some implementations of the current subject matter;

FIG. 29 illustrates an exemplary graphical user interface executed by the application to display recommended recipes within the challenge program shown in FIG. 28, according to some implementations of the current subject matter;

FIG. 30 illustrates an exemplary graphical user interface executed by the application to display recommended exercises within the challenge program shown in FIG. 28, according to some implementations of the current subject matter;

FIG. 31 illustrates an exemplary graphical user interface executed by the application to display recommended stress management activities within the challenge program shown in FIG. 28, according to some implementations of the current subject matter;

FIG. 32 illustrates an exemplary graphical user interface executed by the application to display recommended human support activities within the challenge program shown in FIG. 28, according to some implementations of the current subject matter;

FIG. 33 illustrates an exemplary graphical user interface executed by the application to display an electronic store that sells third party devices, such as wearable devices, from which the interaction module can receive values of at least some biomarkers, according to one variation of the current subject matter; and

FIG. 34 illustrates exemplary display screens of the application on some types of computing devices, such as a particular smartphone, according to some implementations of the current subject matter.

Like reference symbols in various drawings indicate like elements.

### DETAILED DESCRIPTION

A computing system is described that can receive values of a plurality of biomarkers from an application executed on a computing device of a user, generate a score for each biomarker, compute an overall heath score for a user by weighting and adding all biomarker scores, compute a severity associated with each biomarker, identify a physiological condition associated with the severity of each biomarker, generate an explanation of the severity of the biomarker and at least one treatment recommendation based on the severity associated with each biomarker, and send the following to the application executed on the computing device of the user via a communication network: the score for each biomarker, the overall health score, the explanation of the severity, and the at least one treatment recommendation.

The at least one physiological condition can be at least one of: a cardiovascular disease, diabetes, hypertension, obesity, and other conditions. In some implementations, some of such other conditions can include unhealthy diet, consumption of alcohol, lack of exercise, and the like. The at least one treatment recommendation can: 1) prevent or reduce disease progression within the user and the development of disease complications within the user, 2) reverse the disease or its complications within the user, and 3) reduce the need for medications the user is already taking for his/her physiological condition.

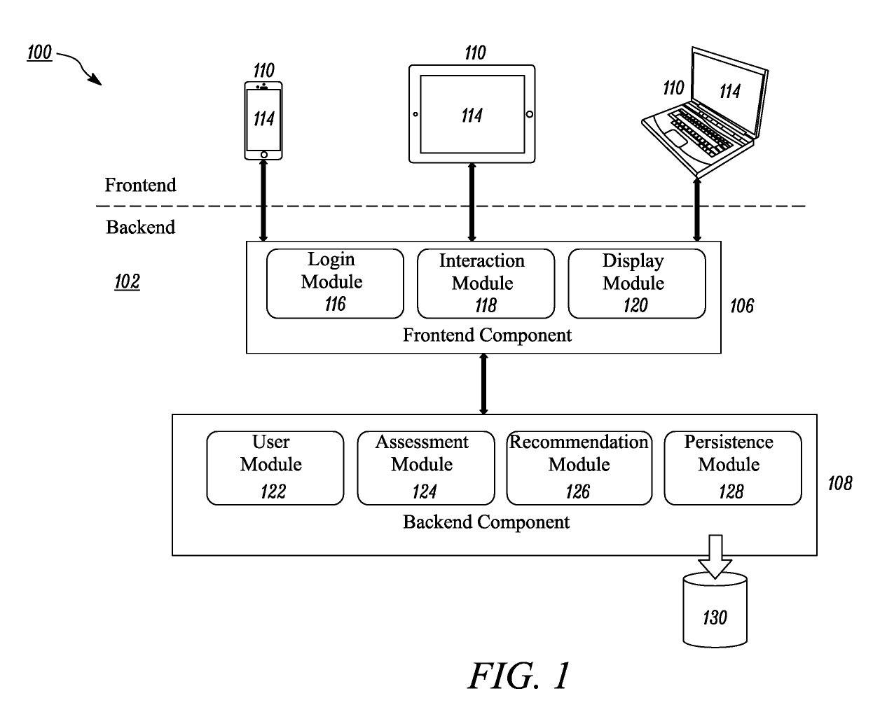

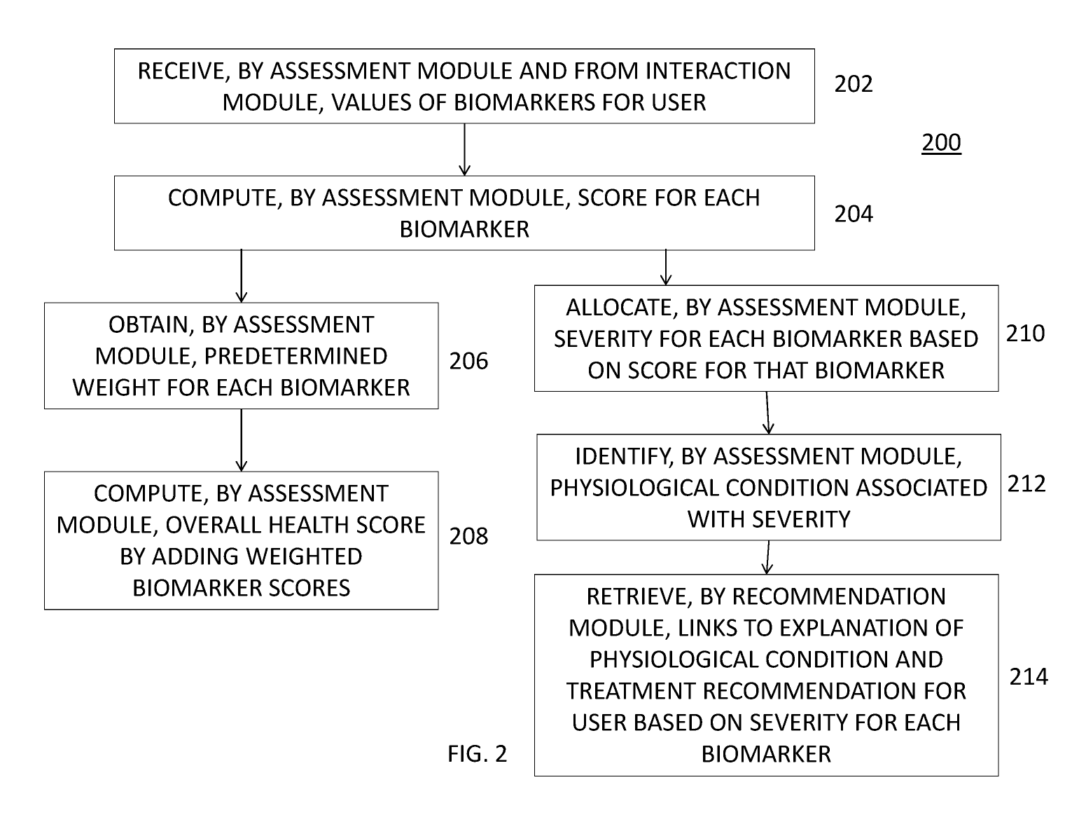

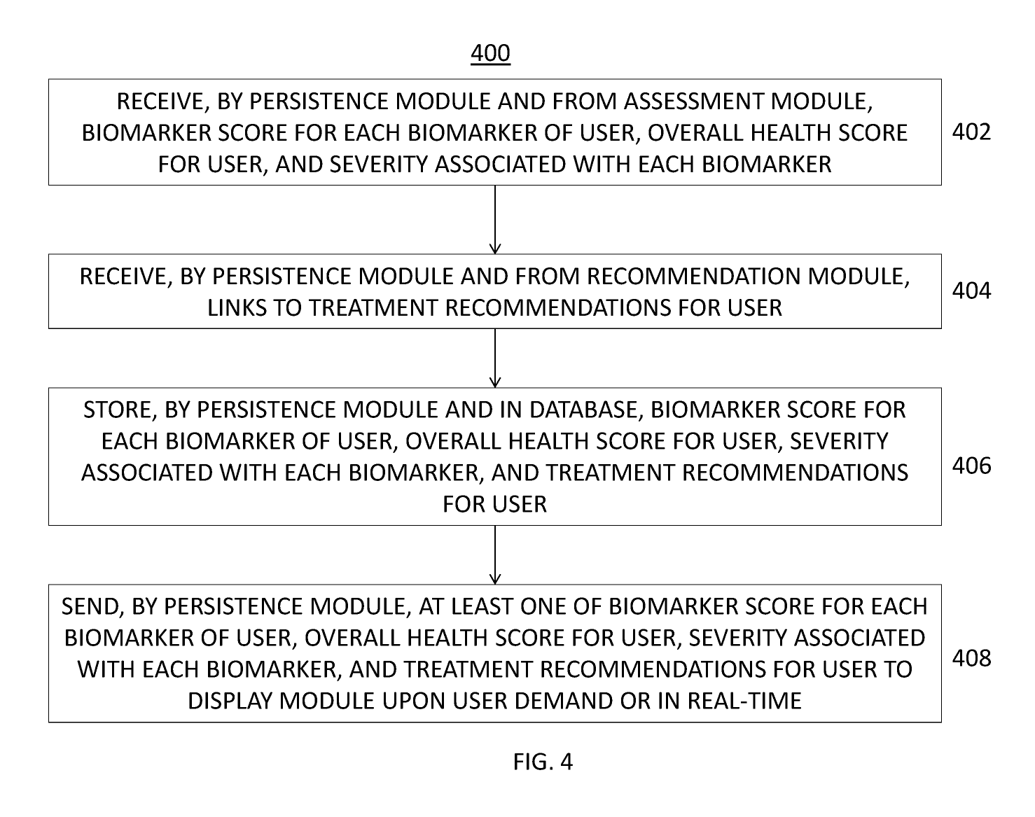

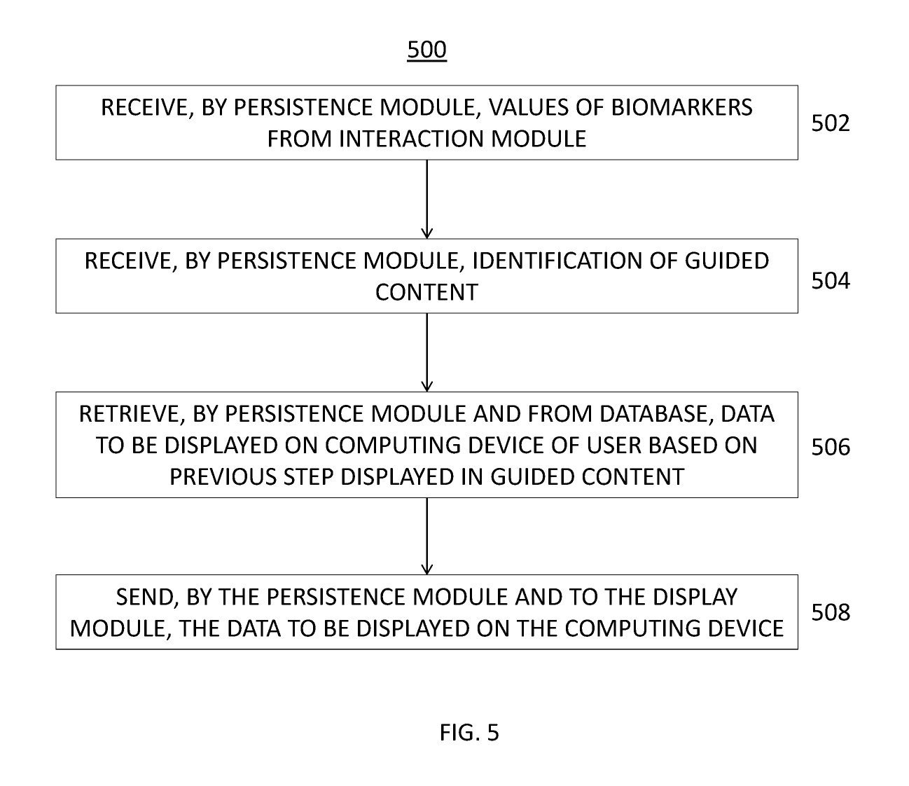

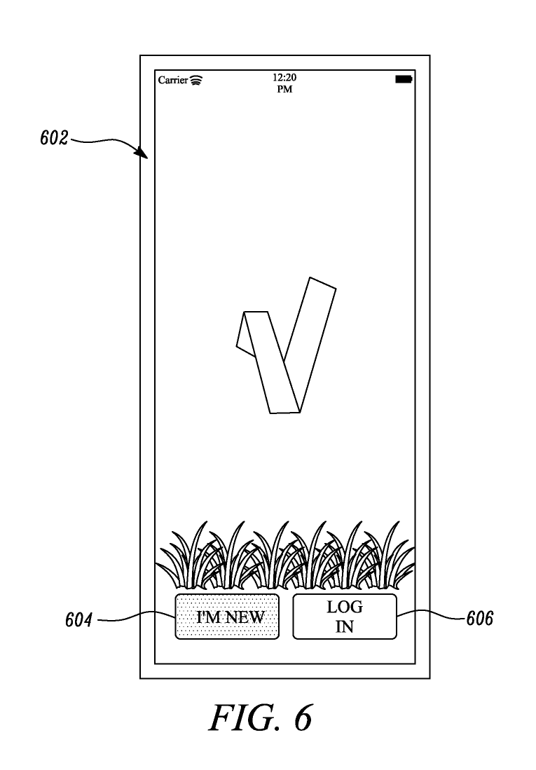

A computing architecture of the computing system is discussed below by FIG. 1. A method of generating the overall health score and the treatment recommendations is discussed below by FIG. 2. Operations of specific modules within the architecture of the computing system of FIG. 1 are described below and shown in FIGS. 4-5. Exemplary graphical user interfaces generated by the computing system are described below and shown in FIGS. 6-34.

I. System For Providing Treatment Recommendations

FIG. 1 is a system diagram illustrating a computer-architecture 100 of a system 102 generating an overall health score of a user and treatment recommendations for the user based on values of biomarkers specific to that individual, according to some implementations of the current subject matter. The system 102 can be located at the backend 104, and can include a frontend component 106 and a backend component 108. The frontend component 106 can communicate with computing devices 110 of one or more users located at the frontend 112 via a communication network. Each computing device 110 can execute an application 114 that can receive values of biomarkers from the user, and display treatment recommendations generated by the system 102.

The frontend component 106 can include a login module 116, an interaction module 118, and a display module 120. The login module 116 can allow a user to: register on the application 114 by creating an account with at least a username and/or a password, enter the username and password on the application 114, and reset the password. The interaction module 118 can receive values of biomarkers input by the user on the application 114. The display module 120 can send data for display to a user from the backend component 108 to the application 114 executed on the computing device 110 of that user.

The backend component 108 can include a user module 122, an assessment module 124, a recommendation module 126, and a persistence module 128. The persistence module 128 can be operably coupled to a database 130. The user module 122 can authenticate a user based on the data received by the user module 122 from the login module 116. The assessment module 124 can receive values of the biomarkers from the interaction module 118, and can use those values to: compute a score (which can also be referred to as a biomarker score) for each biomarker, generate an overall health score by adding weighted biomarker scores for different biomarkers, and determine a severity for each biomarker based on the biomarker score for that biomarker.

A biomarker score for each biomarker can be generated as follows. The assessment module 124 can identify a range within a plurality of ranges within which each biomarker's value lies. The assessment module 124 can then determine, using a table stored in the database, a biomarker score associated with the determined range. Different biomarkers can have different ranges. Each biomarker's score can vary between zero and one hundred, and can be evenly distributed across each range. Some exemplary ranges for exemplary biomarkers can be as follows.

The ranges for an A1C biomarker can be: 0-4.99; 5.0-5.39; 5.4 5.59; 5.6-6.09; 6.1-6.59; 6.6-7.19; 7.2-7.59; 7.6-8.09; 8.1-8.59; 8.6-9.09; and 9.1-9.59. The ranges for activity biomarker can be: 3+ hard; 1-2 hard; 30-45 hard; 5+ light; 3-4 light; 1-2 light; and >1 Light. The ranges for alcohol biomarker can be: 0; 1-2; 3-5; 6-8; 9-12; 13-15; 16-21; and 22+. The ranges for the LDL cholesterol biomarker can be: 0-79; 80-89; 90-99; 100-109; 110-119; 120-129; 130-139; 140-149; 150-159; 160-169; 170-179; and 180+. The ranges for LDL cholesterol medications can be: 0 Medications; 1-2 Medications; and 3+ Medications.

The ranges for nutrition biomarkers can vary based on the nutrition, as follows. When the nutrition is beans, berries, mushrooms, nuts, onions and tomatoes, the ranges can be: 7+; 4-6; 1-3; and 0. When the nutrition is green vegetables, the ranges can be: 14+; 7-13; 1-6; and 0. When the nutrition is high glycemic, the ranges can be: 0; 1-3; 4-6; 7-13; 14-20; and 21+.

The ranges for the smoking biomarker can be: 0; 1-4; 5-9; 10-14; 15-19; 20-24; 25-29; 30-34; 35-39; and 40+. The ranges for the smoking years quit biomarker can be: 0-1; 2-9; 10-15; and 16+. The ranges for the vitamin D biomarker can be: 30− infinity; 25-29; 20-24; 15-19; 10-14; and 0-9. The ranges for the waist to height biomarker can be: 0-0.379; 0.38-0.459; 0.46 0.499; 0.50-0.519; 0.52-0.539; 0.54-0.559; 0.56-0.579; 0.58-0.599; 0.60-0.619; 0.62-0.639; 0.64-0.659; and 0.66-1. The ranges for the systolic blood pressure biomarker can be: 175+; 170-174; 165-169; 160-164; 155-159; 150-154; 145-149; 140-144; 135-139; 130-134; 125-129; 120-124; and 0-120. The ranges for the systolic blood pressure medications can be: 0 medications; 1-2 medications; and 3+ medications.

The severity for each biomarker can be normal, mild, moderate and severe. For each biomarker in the aforementioned example, healthy can correspond to the biomarker score of 76-100, mild (that is, mild risk of developing one or more physiological conditions associated with that particular biomarker) can correspond to the biomarker score of 51-75, moderate (that is, moderate risk of developing one or more physiological conditions associated with that particular biomarker) can correspond to the biomarker score of 26-50, and severe (that is, severe risk of developing one or more physiological conditions associated with that particular biomarker) can correspond to the biomarker score of 0-25. In other implementations, any other suitable range for each of the following severities for each biomarker can be used: normal, mild, moderate and severe. In another implementation, there can be any number of severities rather than four, as noted above.

The recommendation module 126 can receive the severity for each biomarker, identify a physiological condition based on each severity, retrieve from the database 130 and via the persistence module 128 data (for example, a web link or any other URL to a video, which may be available online) characterizing an explanation of the physiological condition for each biomarker, and generate a treatment recommendation for each biomarker based on the severity for that biomarker.

The treatment recommendations can be behavioral changes recommended for the user. For instance, each severity of each biomarker can be associated with a separate treatment recommendation, which, in one example, can be a video that recommends one or more behavioral changes to improve the biomarker value and thus the biomarker score for the user. In one example, the treatment recommendations for mild hypertension may be less severe than those for severe hypertension. The display module 120 can send the treatment recommendations to the computing device 110 of the user in a format based on a communication preference specified by the user on the application 114. The format can be one or more of: a text message, an email, a telephone call, an online indication, a social networking message, and the like. The system 102 can generate the treatment recommendations instantly. The system 102 can make them available constantly (for example, twenty four hours a day, seven days a week, and every day of the year) on the application 114 at the computing devices 110. The instant generation of the recommendations refers to the generation of those recommendations immediately after the one or more values for the one or more biomarkers is received. The terms immediately or immediately after can refer to a time gap of up to 0.1 second. In an alternate implementation, this time gap can be up to 1 second. In a yet another implementation, this time gap can be up to 5 seconds. In an alternately implementation, this time gap can be up to 20 seconds or more.

The persistence module 128 can retrieve data stored in the database 130 when requested by any other module. The database 130 can store some or all of the data associated with the application 114, such as: username, password, biomarker scores, overall health score, treatment recommendations, physiological conditions, links to data explaining those physiological conditions (for example, web links to videos explaining those physiological conditions) of the user; weights associated with each biomarker that are used, as noted above, to calculate the overall health score; entire health data of each user of the application 114; and/or any data used for the operations of the a frontend component 106 and/or the backend component 108.

The frontend component 106 and the backend component 108 can be a part of a model view controller (MVC) architecture. The frontend component 106 can also be referred to as a frontend of the MVC architecture, and the backend component 108 can also be referred to as a backend of the MVC architecture. The MVC architecture is architecturally advantageous over standard JavaScript, as the MVC architecture enables a better organized code, thus an easier to comprehend and easier to maintain code. The MVC architecture can enable a better organized code, as this architecture separates the view logic, as enabled by the frontend component 106, from the business logic, as enabled by the backend component 108. In an alternate implementation, however, the frontend component 106 and the backend component 108 can be a part of a JavaScript.

The computing device 110 can include software, hardware, and any combination thereof. The computing device 110 can be at least one of: a laptop computer, a desktop computer, a tablet computer, a cellular telephone, a smartphone, a phablet, a computing kiosk and any other computing device. The communication network via which the frontend component 106 communicates with computing devices 110 can include one or more of: a local area network, a metropolitan area network, a wide area network, virtual local area network, internet, intranet, Bluetooth network, infrared network, and/or other communication networks. The application 114 can be a software application.

In one implementation, each module (including login module 116, interaction module 118, display module 120, user module 122, assessment module 124, recommendation module 126, and/or persistence module 128) can be a portion of the software code that performs operations executed by that module. Each module can be a cluster of instances such as EC2 instances. Each EC2 instance can be a virtual server in AMAZON's Elastic Compute Cloud (EC2) for running applications on the AMAZON WEB SERVICES (AWS) infrastructure. Each instance can be scaled and deployed independently of other instances. Each module in the frontend component 106 and the backend component 108 can be communicatively coupled to all other modules in the frontend component 106 and the backend component 108.

Although each module (including login module 116, interaction module 118, display module 120, user module 122, assessment module 124, recommendation module 126, and/or persistence module 128) is described as a cluster of instances, in an alternate implementation each module can be a hardware server, which is a physical device. The hardware server or the physical device can be a co-located server, an on-premise server, a collection of servers, any other type of servers, and/or any combination thereof. Different hardware servers can be communicatively coupled using at least one of the following a wireless network, a wired network, a metropolitan area network, a wide area network, a local area network, a virtual local area network, and/or any other type of network and/or any combination thereof.

II. Biomarkers

The biomarkers for which respective values are requested by the application 114 on a graphical user can include at least one of: cholesterol level (for example, low density lipoprotein (LDL) levels or high density lipoprotein (HDL) levels), waist to height ratio, blood pressure, serum A1C levels, alcohol consumption, glycemic food intake, nutrient dense food intake, physical activity level, smoking, Vitamin D level any other biomarker, and any combination thereof.

Elevated levels of cholesterol, in particular LDL and triglycerides in the blood, have been associated with the development of fatty plaques, which can lead to generalized vascular damage, atherosclerosis and eventually heart attack. The term cholesterol, as used herein, refers to the monohydric alcohol form, which is a white, powdery substance that is found in all animal cells and in animal-based foods (not in plants). The term lipoproteins, as used herein, refers to protein spheres that transport cholesterol, triglyceride, or other lipid molecules through the bloodstream. Lipoproteins are categorized into types according to size and density. They can be further defined by whether they carry cholesterol (high density lipoproteins (HDL) and low density lipoproteins (LDL)) or triglycerides (intermediate density lipoproteins (IDL), very low density lipoproteins (VLDL), and chylomicrons)). Atherosclerosis is a leading form of cardiovascular disease, which involves the slow build-up of fatty plaques on the arterial wall. This build-up can damage the vascular endothelium causing inflammation, a narrowing of the arteries and potential arterial blockages that can result in heart attacks. Cholesterol levels in many people can be controlled by diet, but for many patients diet changes alone are insufficient to reduce high cholesterol. Cholesterol lowering drugs such as Zocor® (simvastatin) and Lipitor® (atorvastatin) can be prescribed to help patients lower their cholesterol levels. Serum cholesterol levels (such as LDL levels) can be measured by any means known in the art. Cholesterol is typically measured as milligrams per deciliter (mg/dL) of blood in the United States and some other countries. In the United Kingdom, most European countries, and Canada, millimoles per liter of blood (mmol/L) is the most commonly used measure.

Blood pressure (BP) is the pressure exerted by circulating blood upon the walls of blood vessels. As used herein, the term blood pressure refers to the arterial pressure in the systemic circulation. Blood pressure is usually expressed in terms of the systolic (maximum) pressure over diastolic (minimum) pressure and is measured in millimeters of mercury (mm Hg). In some implementations, a normal resting systolic (diastolic) blood pressure in an adult is approximately 120 mm Hg (80 mm Hg), abbreviated as 120/80 mm Hg. Blood pressure can be assessed by any means known in the art but is most commonly measured non-invasively via a sphygmomanometer.

A waist-to-height ratio (WHtR), also called waist-to-stature ratio (WSR), is defined as an individual's waist circumference divided by their height, both measured in the same units. The WHtR is a measure of the distribution of body fat. Higher values of WHtR are correlated with a higher risk of obesity-related cardiovascular diseases as well as with abdominal obesity.

Glycated hemoglobin (also known as hemoglobin A1C; sometimes also referred to as being Hb1c or HGBA1C) is a form of hemoglobin that is measured primarily to identify the three-month average plasma glucose concentration. The test is limited to a three-month average because the lifespan of a red blood cell is four months (120 days). Glycated hemoglobin is formed in a non-enzymatic glycation pathway by hemoglobin's exposure to plasma glucose. Normal levels of glucose produce a normal amount of glycated hemoglobin. As the average amount of plasma glucose increases, the fraction of glycated hemoglobin increases in a predictable way. As such, A1C is a biomarker for average blood glucose levels over the previous three months before the measurement. In individuals diagnosed with or predisposed to developing diabetes mellitus, higher A1C indicates poorer control of blood glucose levels and is also associated with conditions such as cardiovascular disease, nephropathy, neuropathy, and retinopathy. Any method known in the art can be used to determine serum A1C levels such as, but not limited to, high-performance liquid chromatography (HPLC), immunoassays, enzymatic assays, capillary electrophoresis, and/or boronate affinity chromatography.

Alcohol consumption refers to the daily intake or consumption of alcoholic beverages and is typically measured via self-reporting. However, alcohol consumption can also be measured using blood tests and/or devices capable of detecting alcohol consumption via an individual's breath (i.e. a breathalyzer).

The term glycemic food intake, as used herein, refers consumption of food or food ingredient and the subsequent effect of that food or food ingredient on blood sugar (glucose), A1C, and/or insulin levels. Whether a food is considered high or low for purposes of glycemic food intake can be determined based on a glycemic index (GI) established for the food. A food's glycemic index is determined relative to the effect of consuming pure glucose. Foods with carbohydrates that break down quickly during digestion and which release glucose rapidly into the bloodstream tend to have a high GI; foods with carbohydrates that break down more slowly, releasing glucose more gradually into the bloodstream, tend to have a low GI. Glycemic food intake is typically self-reported.

The term nutrient dense food intake, as used herein, refers to consumption of food having a relatively high proportion of nutrients relative to other foods. Nutrient-dense foods such as fruits and vegetables are the opposite of energy-dense food (also called empty calorie food), such as alcohol and foods high in added sugar or processed cereals. Further, nutrient-dense foods are good sources of vitamins or minerals such as the B-vitamins, vitamins A, C, D and E, protein, calcium, iron, potassium, zinc, fiber and monounsaturated fatty acids. Nutritional rating systems are methods of ranking or rating food products or food categories to communicate the nutritional density of food in a simplified manner to a target audience. Rating systems have been developed by governments, nonprofit organizations, or private institutions and companies. Such rating systems can be used in accordance with the methods disclosed herein to determine types of nutrient dense foods for determination of nutrient dense food intake.

Physical activity level (PAL) is a way to express a person's daily physical activity as a number, and is used to estimate a person's total energy expenditure. In combination with the basal metabolic rate, it can be used to compute the amount of food energy a person needs to consume in order to maintain a particular lifestyle. In some implementations, physical activity level is defined for a non-pregnant, non-lactating adult as the total energy expenditure (TEE) in a 24-hour period, divided by his or her basal metabolic rate (BMR). Physical activity level is typically self-reported. However, in some implementations, PAL can be determined at least in part from smart and/or wearable devices (for example, APPLE watch, a FITBIT device, etc.), and/or other personal health monitoring devices.

Smoking is one of the most common forms of recreational drug use. Tobacco smoking is the most popular form, being practiced by over one billion people globally, of whom the majority are in the developing world. Smoking behavior and frequency is typically a self-reported biomarker in accordance with the methods disclosed herein.

A Vitamin D level can refer to the amount of vitamin D in the human body. This amount of vitamin D in the human body can be determined using a blood test, such as a 25-hydroxy vitamin D blood test. In one implementation, a level of 30 nanograms/milliliter (also referred to as 30 ng/mL) to 50 ng/mL can indicate that the human being is healthy with respect to vitamin D, and a level less than 12 ng/mL can indicate that the human being has a vitamin D deficiency.

A “biomarker” used herein refers to any measurement related to the biological system of an individual being assessed and/or treated. It can include, but is not limited to, measurement of molecules (for example, proteins, serum cholesterol levels) in a sample from such individual, information provided by an individual (for example, age, height, waist size, blood pressure, etc.) and actions that the individual takes (for example, consumption of certain foods, physical activity, etc.).

III. Physiological Conditions Associated with Biomarkers

As shown in Table 1, each of the biomarkers assessed in accordance with the methods described herein maps to or is associated with one or more physiological conditions that adversely affect health.

TABLE 1

| Biomarker | Physiological Condition |
|---|---|
| Physical Activity Level | Poor activity; sedentary lifestyle |
| Cholesterol (for example, | Cardiovascular disease |
| LDL or HDL) | |
| A1C | Diabetes (such as type 2 diabetes); |
| | pre-diabetes; metabolic syndrome |
| Alcohol | Excessive alcohol consumption |
| Blood pressure (for example, | Hypertension |
| systolic and/or diastolic) | |
| High glycemic food intake | Poor nutrition |
| Low nutrient dense food intake | Poor nutrition |
| Waist to height ratio | Obesity |
| Smoking | Smoking-related illness |
| Vitamin D | Low Vitamin D |

A lack of physical activity is one of the leading causes of preventable death worldwide. As used herein, individuals who have no or irregular physical activity are said to be engaging in a “sedentary lifestyle.” Lack of exercise causes muscle atrophy, i.e. shrinking and weakening of the muscles and accordingly increases susceptibility to physical injury. Additionally, regular physical activity is correlated with immune system function and decreased development of cardiovascular and endocrine-related disorders.

“Cardiovascular disease,” as used herein, refers to a class of diseases that involve the heart or blood vessels and can include coronary artery diseases (CAD) such as angina and myocardial infarction (commonly known as a heart attack). Complications associated with cardiovascular diseases can include, without limitation, stroke, hypertensive heart disease, rheumatic heart disease, cardiomyopathy, heart arrhythmia, congenital heart disease, valvular heart disease, carditis, aortic aneurysms, peripheral artery disease, and venous thrombosis. Cardiovascular diseases are the leading cause of death globally (Mendis et al., World Health Organization (2011). Global Atlas on Cardiovascular Disease Prevention and Control. World Health Organization in collaboration with the World Heart Federation and the World Stroke Organization. pp. 3-18).

Diabetes mellitus (DM), commonly referred to as diabetes, is a group of metabolic diseases in which there are high blood sugar levels over a prolonged period. Symptoms of high blood sugar include frequent urination, increased thirst, and increased hunger. If left untreated, diabetes can cause complications which can include, without limitation, diabetic ketoacidosis, nonketotic hyperosmolar coma, or death. Serious long-term complications include heart disease, stroke, chronic kidney failure, foot ulcers, and damage to the eyes (for example, retinopathy). In some implementations, the type of diabetes is type 2 diabetes. Type 2 diabetes begins with insulin resistance, a condition in which cells fail to respond to insulin properly. As the disease progresses a lack of insulin production by the pancreas may also develop. The primary cause of type 2 diabetes is excessive body weight and not enough physical activity.

The long-term effects of alcohol consumption range from cardio-protective health benefits for low to moderate alcohol consumption in industrialized societies to higher rates of cardiovascular disease to severe detrimental effects in cases of chronic alcohol abuse. Complications associated with large levels of alcohol intake include an increased risk of alcoholism, malnutrition, chronic pancreatitis, alcoholic liver disease and cancer. In addition, damage to the central nervous system and peripheral nervous system can occur from chronic alcohol abuse. The long-term use of alcohol is capable of damaging nearly every organ and system in the body.

Consistently high blood pressure is known as hypertension. High blood pressure usually does not cause symptoms. However, long term high blood pressure, is a major risk factor for complications such as, without limitation, coronary artery disease, stroke, heart failure, peripheral vascular disease, vision loss, and chronic kidney disease.

As used herein, “poor nutrition” refers to consistent consumption of both foods with a high glycemic index as well as low nutrient density. Chronic consumption of a diet with a high glycemic index is independently associated with complications such as increased risk of developing type 2 diabetes, cardiovascular disease, and certain cancers. Further, nutritional deficiencies (such as, but not limited to, vitamin and mineral deficiencies), are associated with a number of diseases and conditions as well as a predisposition for developing cardiovascular diseases and/or diabetes.

“Obesity,” as used herein refers to a medical condition in which excess body fat has accumulated to the extent that it has a negative effect on health. Complications associated with excessive body weight include cardiovascular diseases, diabetes mellitus type 2, obstructive sleep apnea, certain types of cancer, osteoarthritis, and asthma. As a result, obesity has been found to reduce life expectancy. Obesity is most commonly caused by a combination of excessive food intake, lack of physical activity, and genetic susceptibility.

Smoking generally has negative health effects, because smoke inhalation inherently poses challenges to various physiologic processes such as respiration. Diseases and complications related to tobacco smoking have been shown to kill approximately half of long term smokers when compared to average mortality rates faced by non-smokers.

IV. Generating Overall Health Score and Treatment

Recommendation(s)

FIG. 2 is a flow diagram illustrating an exemplary process 200 for generating (which can also be referred to as computing, determining or calculating) an overall health score for the user, and generation of treatment recommendations for the user, according to some implementations of the current subject matter. The assessment module 124 can receive, at 202 and from the interaction module 118, values of biomarkers for the user that are either input on the application 114 on the computing device 110. The assessment module 124 can computer, at 204, a score (also referred to as a biomarker score) for each biomarker. The assessment module 124 can obtain, at 206, a predetermined weight for each biomarker. The assessment module 124 can compute, at 208, an overall health score by: retrieving weights for each biomarker score, and adding weighted biomarker scores to attain the overall health score.

The assessment module 124 can allocate, at 210, a severity for each biomarker based on the biomarker score for that biomarker. The assessment module 124 can identify, at 212, at least one physiological condition associated with the severity. The recommendation module 126 can retrieve, at 214, based on the severity and from the database 130 via the persistency module 128, at least one link—such as a uniform resource location (URL), which can be a web URL—to: (1) an explanation of the at least one physiological condition, and (2) at least one treatment recommendation for the user. The assessment module 124 can subsequently send the retrieved at least one link to the explanation of the at least one physiological condition along with the at least one treatment recommendation to the display module 120, which can enable the display of the aforementioned explanation and the at least one treatment recommendation on the computing device 110.

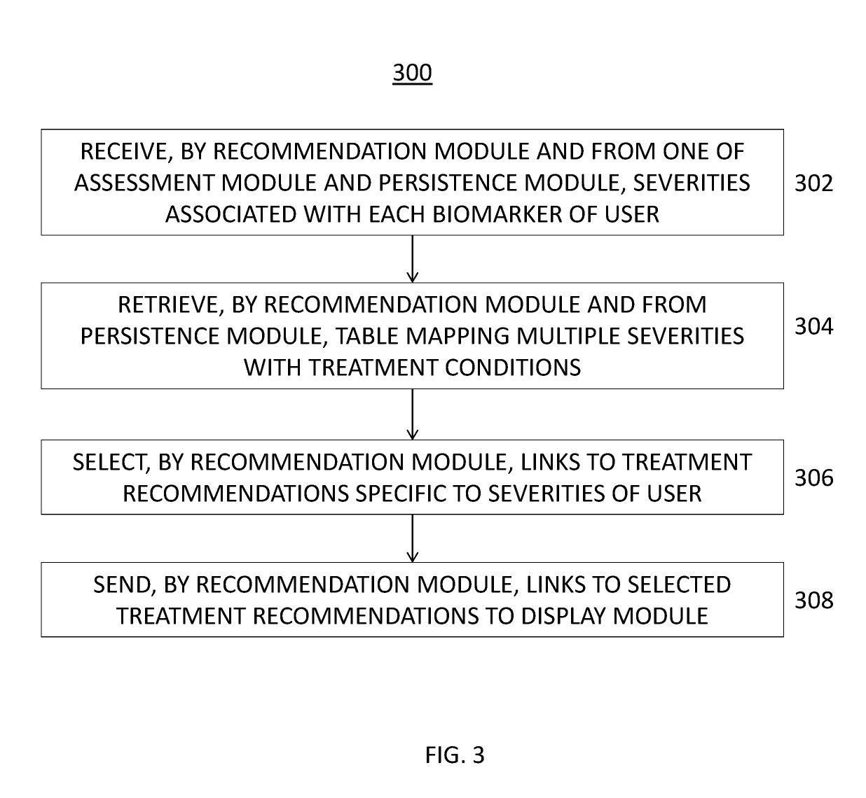

FIG. 3 is a flow diagram illustrating an exemplary process 300 for providing treatment recommendations using the recommendation module 126, according to some implementations of the current subject matter. The recommendation module 126 can receive, at 302 and frim one of the assessment module 124 (when treatment recommendations are being made in real-time) and persistence module 128 (when treatment recommendations are alternately being made after large lapses/delays in time), severities associated with each biomarker of the user. The recommendation module 126 can retrieve, at 304 and from the persistence module 128, a table mapping multiple severities with links to treatment recommendations. The recommendation module 126 can select, at 306, links to treatment recommendations specific to severities for the biomarkers of the user. The recommendation module 126 can send, at 308, the links to the selected treatment recommendations to the display module 120, which can enable the display of the treatment recommendations for the user on the computing device 110.

FIG. 4 is a flow diagram illustrating a process 400 for using the persistence module 128 to provide at least one of biomarker score for each biomarker of user, overall health score for user, severity associated with each biomarker, and treatment recommendations for user to display module upon user demand or in real-time, according to some implementations of the current subject matter. The persistence module 128 can receive, at 402 and from the assessment module 124, an overall health score for the user and a severity associated with each biomarker. The persistence module 128 can receive, at 404 and from the recommendation module 126, links to treatment recommendations for the user. The persistence module 128 can store, at 406 and in the database 130, the biomarker score for each biomarker of the user, the overall health score of the user, the severity associated with each biomarker of the user, and links to treatment recommendations for the user. The persistence module can send, at 408 and to the display module 120 either upon user demand or in real-time, at least one of the biomarker score for each biomarker of the user, the overall health score of the user, the severity associated with each biomarker of the user, and links to treatment recommendations for the user.

In an alternate implementation, the assessment module 124 may directly send to the display module 120 at least one of: biomarker score for each biomarker of individual, overall health score for individual, and severity associated with each biomarker. This direct sending by the assessment module can prevent delays or lag time caused by first storing data in the database 130. In this implementation, the data can still be stored in the database 130, but such storage can be performed simultaneously with or after the sending of a copy of the data by the assessment module 124 to the display module 120.

In one implementation, the recommendation module 126 may directly send to the display module 120 the treatment recommendations. In this implementation, the data can still be stored in the database 130, but such storage can be performed simultaneously with or after the sending of a copy of the data by the assessment module 126 to the display module 120.

FIG. 5 is a flow diagram illustrating an exemplary process 500 for using the persistence module 128 to send data (for example, guided content, which can include data within challenge programs) for display by the application 114 executed on the computing device 110, according to some implementations of the current subject matter. The persistence module 128 can receive, at 502, values of biomarkers from the interaction module 118. The persistence module 128 can receive, at 504, an identification of guided content. The guided content can be one or more challenge programs, which can guide a user in a timely (for example, a weekly, daily, monthly, or the like) fashion as to what further steps/activities to undertake. The persistence module 128 can retrieve, at 506 and from the database 130, data to be displayed on the computing device 110 based on a previous step displayed in the guided content. The persistence module 128 can send, at 508 and to the display module 120, the data to be displayed on the computing device 110. The display module 120 can enable the display of this data on the computing device 110.

V. Computing Application for Generating Treatment Recommendations

FIG. 6 illustrates a graphical user interface 602 executed by the application 114 when a user opens the application 114, according to some implementations of the current subject matter. The graphical user interface 602 can include a first button 604, which the user can select to register as a new user of the application 114, and a second button 606, which the user can select to login using his or her authentication data (for example, username and/or password). The application 114 can send the received authentication data to the login module 116, which can further send that authentication data to the user module 122, which can authenticate the user.

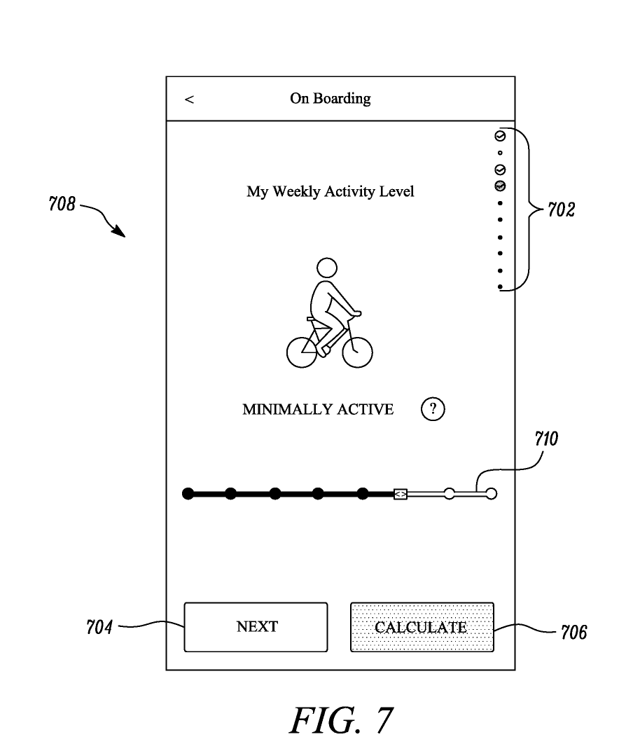

FIGS. 7-19 illustrate exemplary graphical user interfaces executed by the application 114 to receive values of biomarkers for the user. These graphical user interfaces can be presented in the order in which FIGS. 7-19 are presented. In alternate implementations, however, these graphical user interfaces can be presented in any order. These graphical user interfaces of FIGS. 7-12 and 15-19 can have a graphical display feature 702 that can indicate by displaying, using colors and symbols (for example, check marks and dots): biomarkers for which values have been provided by the user; biomarkers for which the user has not provided values but accessed them and skipped providing those values; and biomarkers for which the user has not provided values because the application 114 has not yet displayed graphical user interfaces requesting values for those biomarkers. In an alternate implementation, the graphical feature 702 can display, using one or more colors and/or one or more symbols, a severity associated with each biomarker.

The graphical user interfaces of FIGS. 7-12 and 15-19 include a next button 704, which the user can select to navigate to the next screen without providing or updating the value of the biomarker on the corresponding graphical user interface, and a calculate button 706, which the user can select to initiate the calculation of the biomarker score by the assessment module 124 based on the value provided by the user.

FIG. 7 illustrates an exemplary graphical user interface 708 executed by the application 114 to receive a value of the weekly activity level biomarker of the user, according to some implementations of the current subject matter. The graphical user interface 708 allows the user to select the value of this biomarker using the graphical feature 710. The application 114 can send the value of the biomarker received using the graphical feature 710 to the interaction module 118, which can forward those values to the backend component 108.

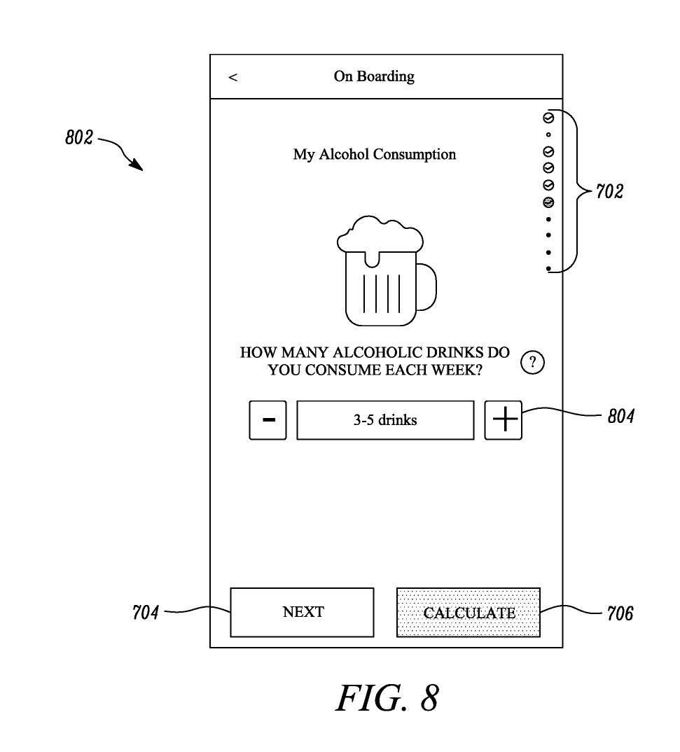

FIG. 8 illustrates an exemplary graphical user interface 802 executed by the application 114 to receive a value of the alcoholic drinks consumed per week biomarker of the user, according to some implementations of the current subject matter. The graphical user interface 802 allows the user to select the value of this biomarker using the graphical feature 804. The application 114 can send the value of the biomarker received using the graphical feature 802 to the interaction module 118, which can forward those values to the backend component 108.

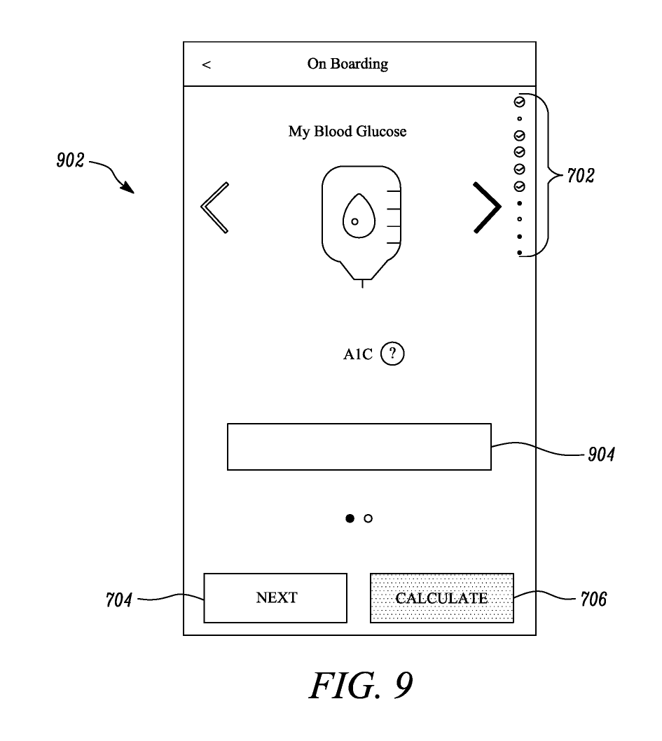

FIG. 9 illustrates an exemplary graphical user interface 902 executed by the application 114 to receive a value of the A1C biomarker of the user, according to some implementations of the current subject matter. The graphical user interface 902 allows the user to provide the value of this biomarker using the graphical feature 904. The application 114 can send the value of the biomarker received using the graphical feature 902 to the interaction module 118, which can forward those values to the backend component 108.

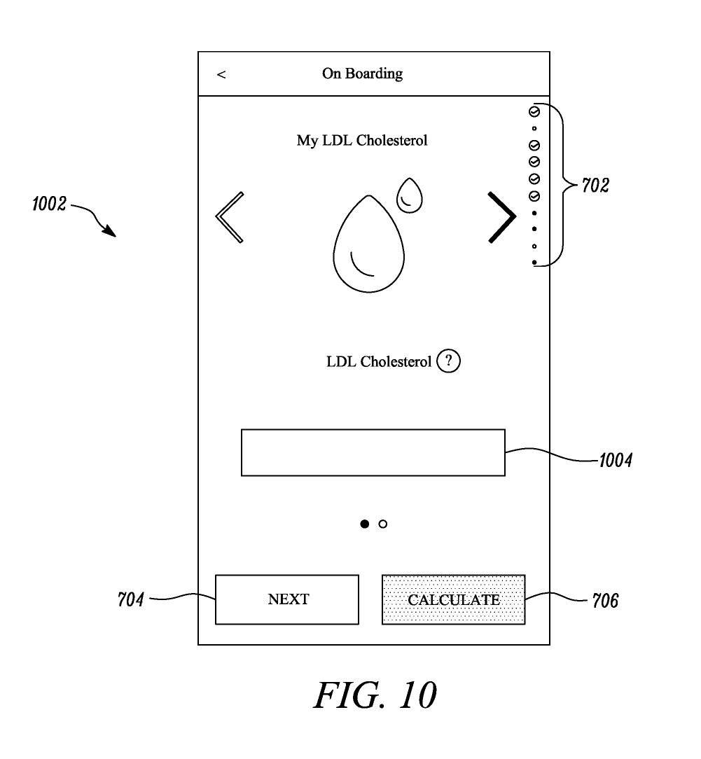

FIG. 10 illustrates an exemplary graphical user interface 1002 executed by the application 114 to receive a value of the LDL cholesterol biomarker of the user, according to some implementations of the current subject matter. The graphical user interface 1002 allows the user to provide the value of this biomarker using the graphical feature 1004. The application 114 can send the value of the biomarker received using the graphical feature 1002 to the interaction module 118, which can forward those values to the backend component 108.

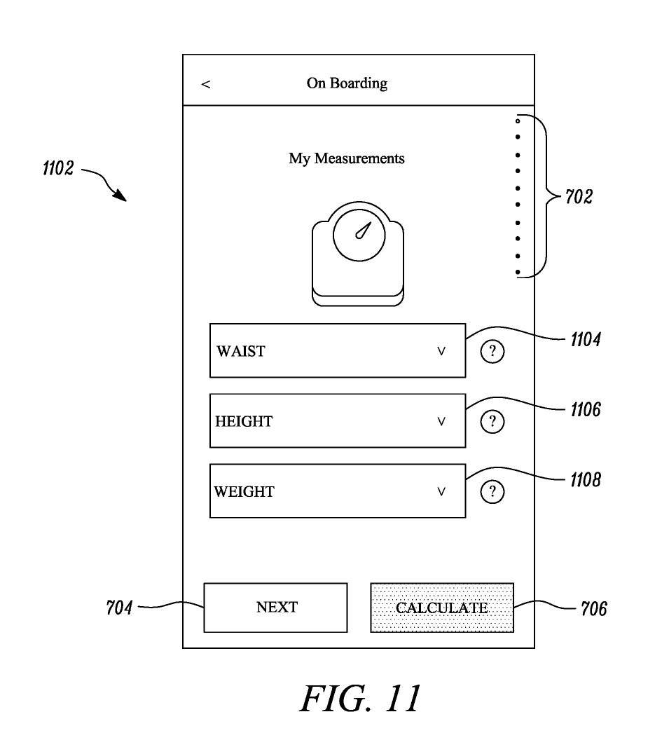

FIG. 11 illustrates an exemplary graphical user interface 1102 executed by the application 114 to receive values of the waist, height, and weight biomarkers of the user, according to some implementations of the current subject matter. The graphical user interface 1002 allows the user to provide the values of these biomarkers using the graphical features 1104, 1106, and 1108, respectively. The application 114 can send the values of the biomarkers received using the graphical features 1104, 1106, and 1108 to the interaction module 118, which can forward those values to the backend component 108.

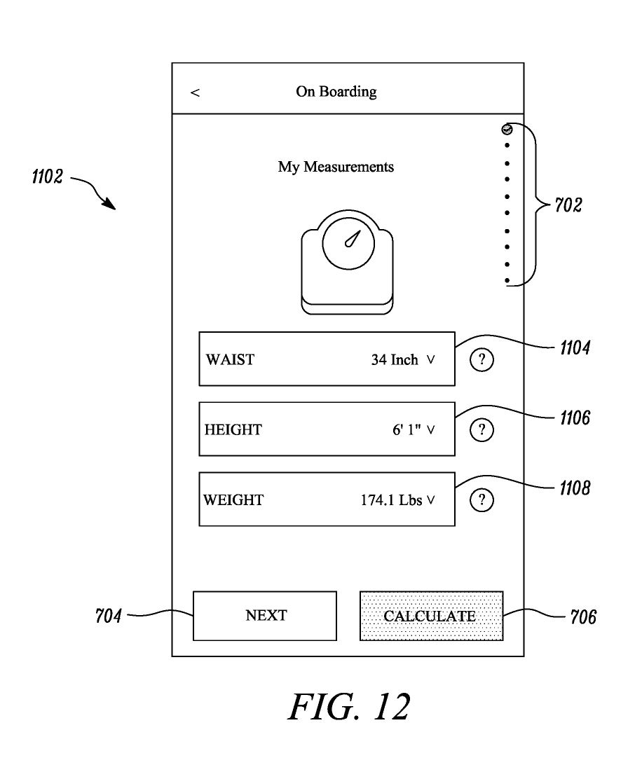

FIG. 12 illustrates the graphical user interface 1102 of FIG. 11 where the graphical features 1104, 1106, and 1108 have been used by the user to input values of the waist, height, and weight biomarkers of the user, according to some implementations of the current subject matter.

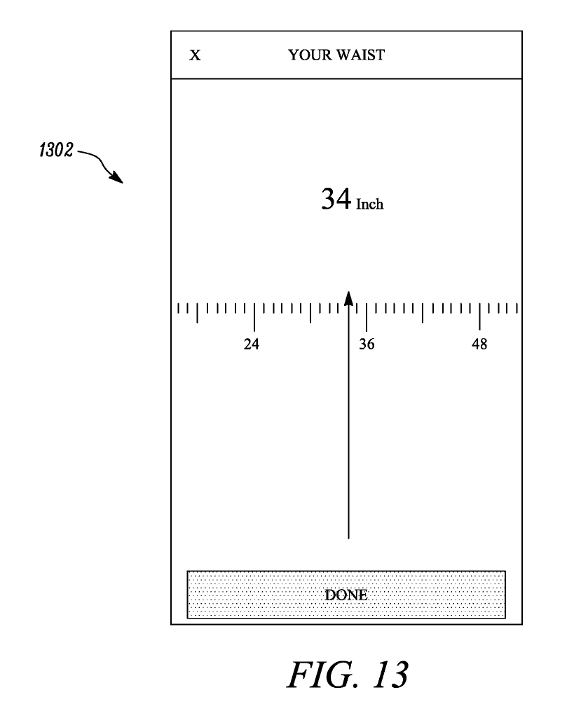

FIG. 13 illustrates an exemplary graphical feature 1302 displayed by the application when the graphical feature 1104 for inputting the waist biomarker is selected in the graphical user interface 1102 of FIG. 11 or 12, according to some implementations of the current subject matter. The graphical feature 1302 can allow the user to graphically select the waist of the user.

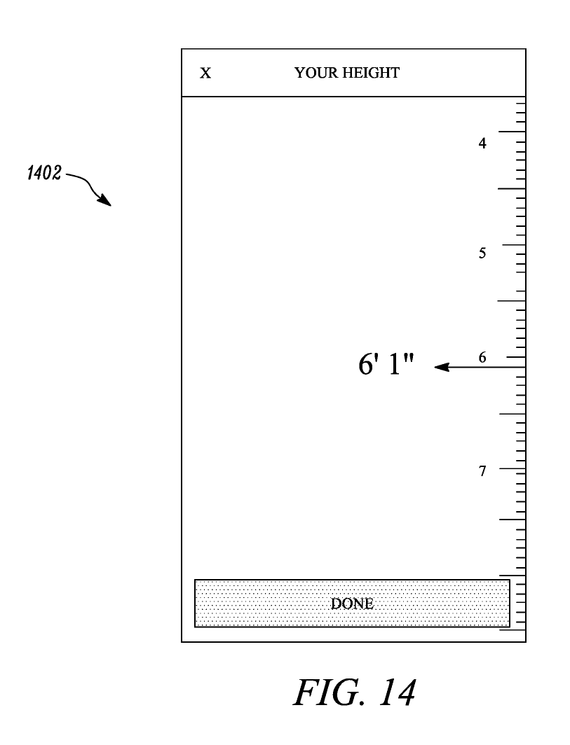

FIG. 14 illustrates an exemplary graphical feature 1402 displayed by the application when the graphical feature 1106 for inputting the height biomarker is selected by the user in the graphical user interface 1102 of FIG. 11 or 12, according to some implementations of the current subject matter. The graphical feature 1402 can allow the user to graphically select the height of the user.

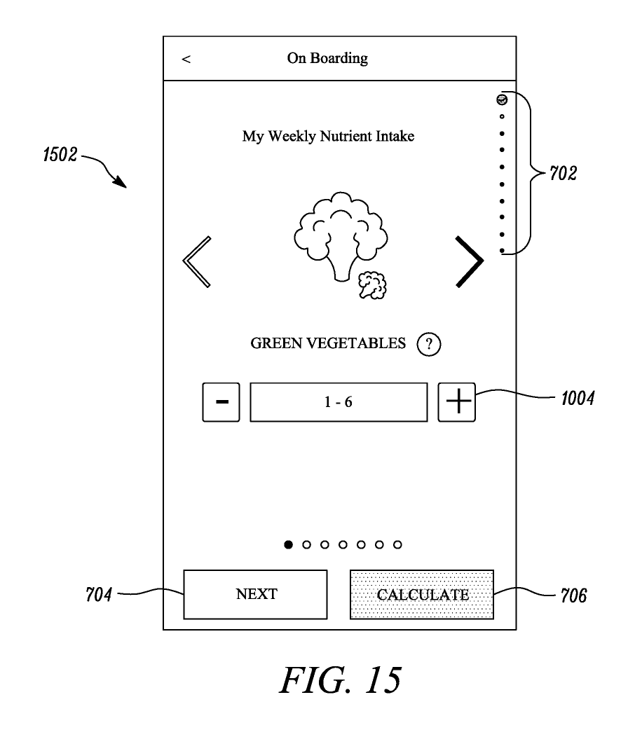

FIG. 15 illustrates an exemplary graphical user interface 1502 executed by the application 114 to receive values of the weekly nutrient intake biomarker of the user, according to some implementations of the current subject matter. The graphical user interface 1502 can allow the user to provide the value of this biomarker using the graphical feature 1504. The application 114 can send the value of this biomarker received using the graphical feature 1504 to the interaction module 118, which can forward that value to the backend component 108.

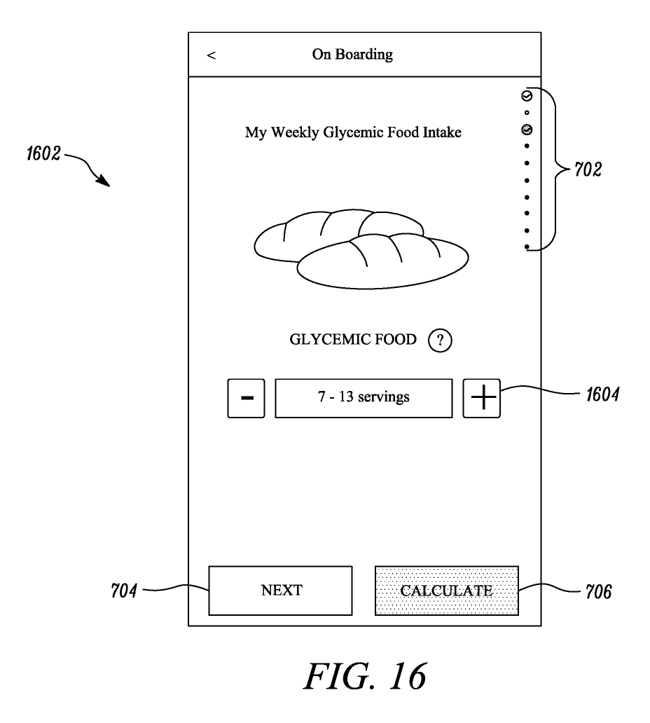

FIG. 16 illustrates an exemplary graphical user interface 1602 executed by the application 114 to receive values of the weekly glycemic food intake biomarker of the user, according to some implementations of the current subject matter. The graphical user interface 1602 can allow the user to provide the value of this biomarker using the graphical feature 1604. The application 114 can send the value of this biomarker received using the graphical feature 1604 to the interaction module 118, which can forward that value to the backend component 108.

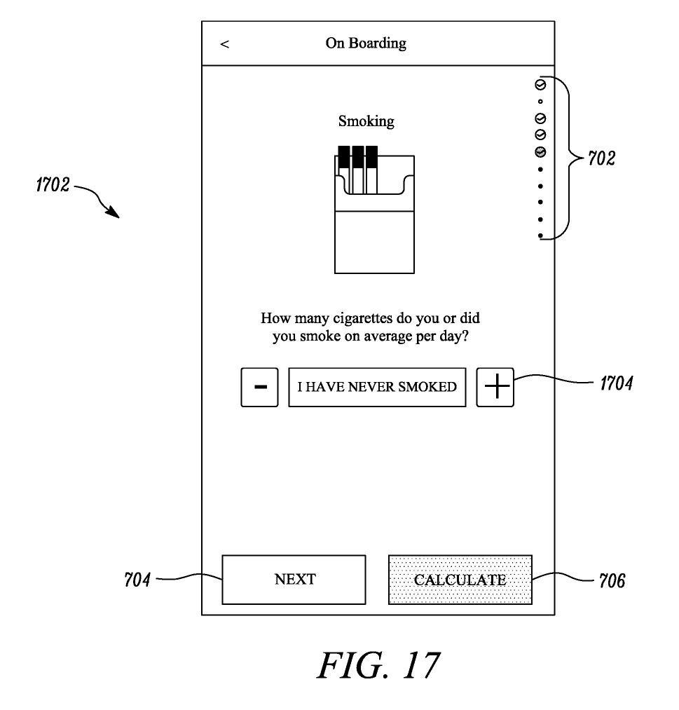

FIG. 17 illustrates an exemplary graphical user interface 1702 executed by the application 114 to receive values of the average number of cigarettes smoked per day biomarker of the user, according to some implementations of the current subject matter. The graphical user interface 1702 can allow the user to provide the value of this biomarker using the graphical feature 1704. The application 114 can send the value of this biomarker received using the graphical feature 1704 to the interaction module 118, which can forward that value to the backend component 108.

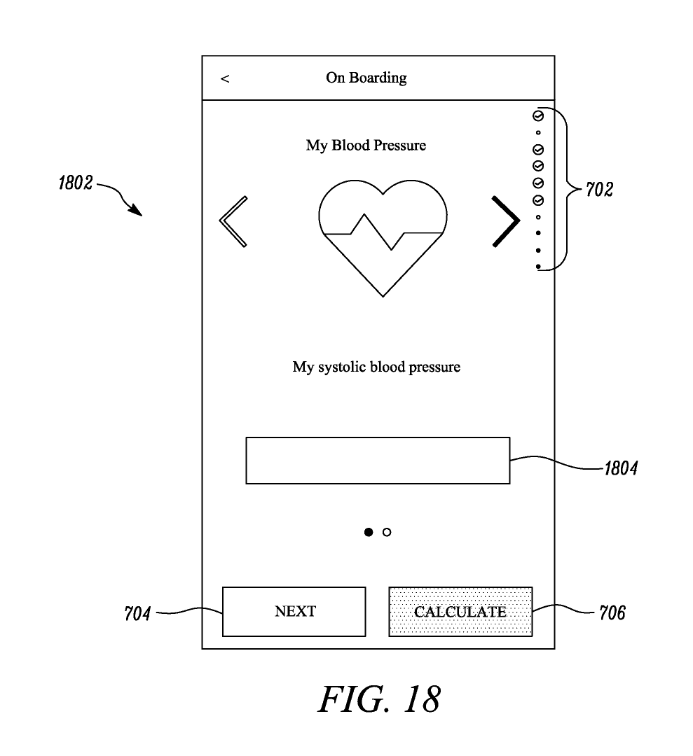

FIG. 18 illustrates an exemplary graphical user interface 1802 executed by the application 114 to receive values of the blood pressure biomarker of the user, according to some implementations of the current subject matter. The graphical user interface 1802 can allow the user to provide the value of this biomarker using the graphical feature 1804. The application 114 can send the value of this biomarker received using the graphical feature 1804 to the interaction module 118, which can forward that value to the backend component 108.

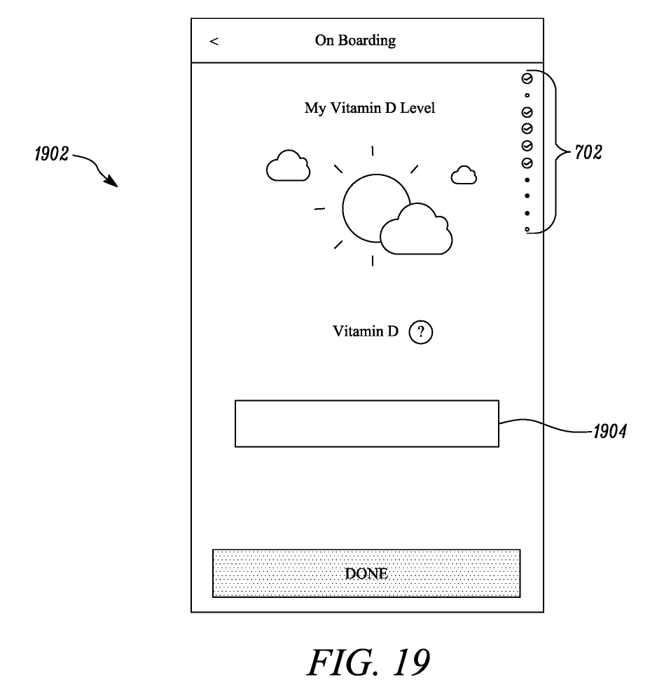

FIG. 19 illustrates an exemplary graphical user interface 1902 executed by the application 114 to receive values of the vitamin D biomarker of the user, according to some implementations of the current subject matter. The graphical user interface 1902 can allow the user to provide the value of this biomarker using the graphical feature 1904. The application 114 can send the value of this biomarker received using the graphical feature 1904 to the interaction module 118, which can forward that value to the backend component 108.

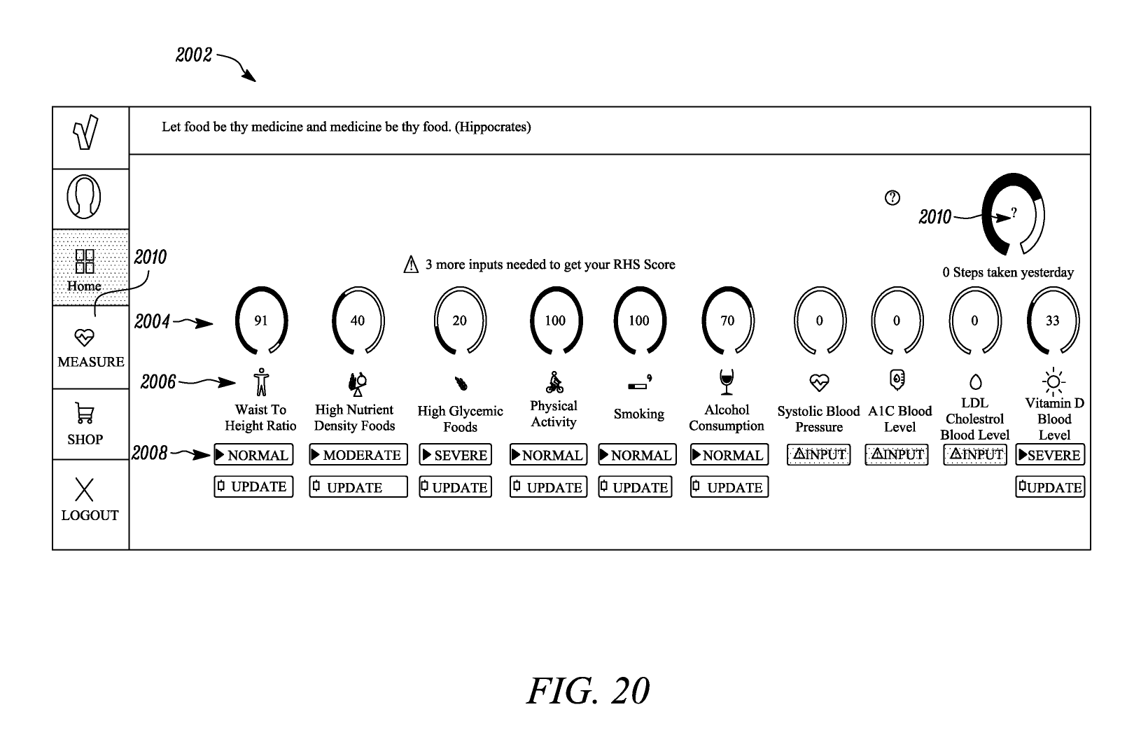

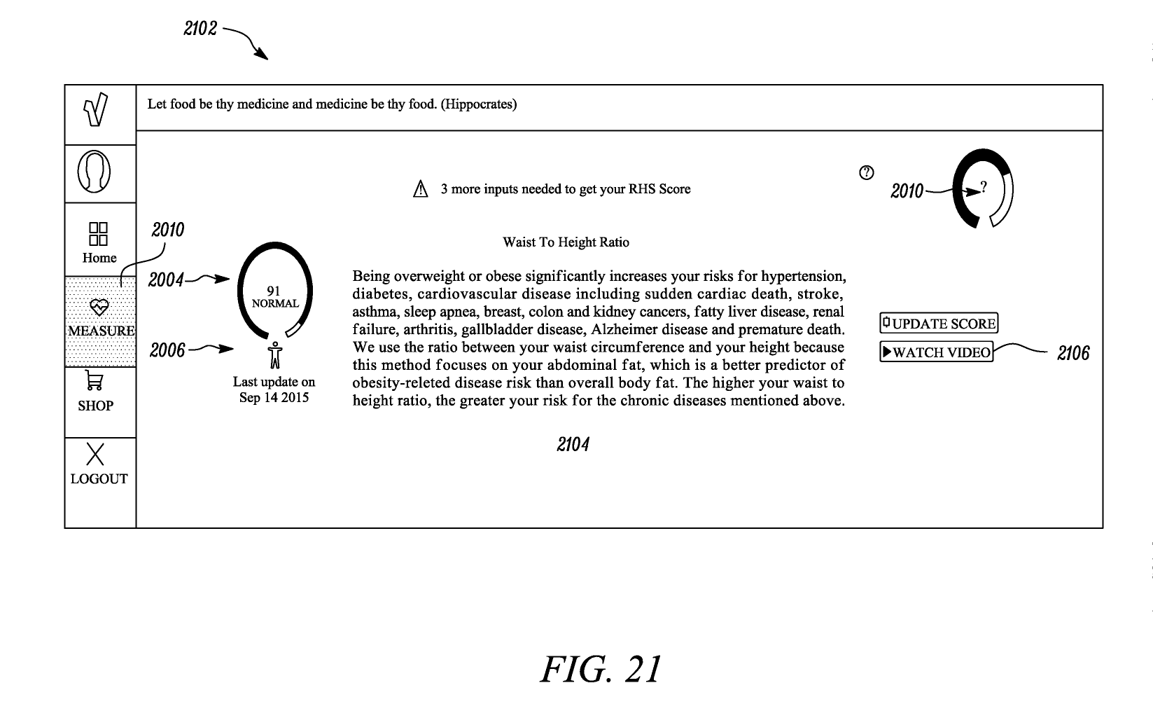

FIG. 20 illustrates an exemplary graphical user interface 2002 executed by the application 114 to display the biomarker score 2004 for each biomarker 2006, a severity 2008 determined for each biomarker, a button 2010 which when selected by the user makes the application 114 display data 2104 (for example, text, as shown in FIGS. 21-26) characterizing an explanation of the severity for specific biomarkers, and an overall health score 2012 when values of a threshold number of biomarkers has been received by the application 114, according to some implementations of the current subject matter. Although the data including the treatment recommendation is described as text, in other implementations other forms of data may additionally or alternately be used, such as: video, audio, and/or any combination thereof.

FIG. 21 illustrates an exemplary graphical user interface 2102 executed by the application 114 to display: data 2104 (for example, text) characterizing an explanation of the severity for the displayed biomarker 2006, and a button 2106 which when selected by the user makes the application 114 display data (for example, video) including a treatment recommendation based on the severity 2008 of that particular biomarker 2006, according to some implementations of the current subject matter.

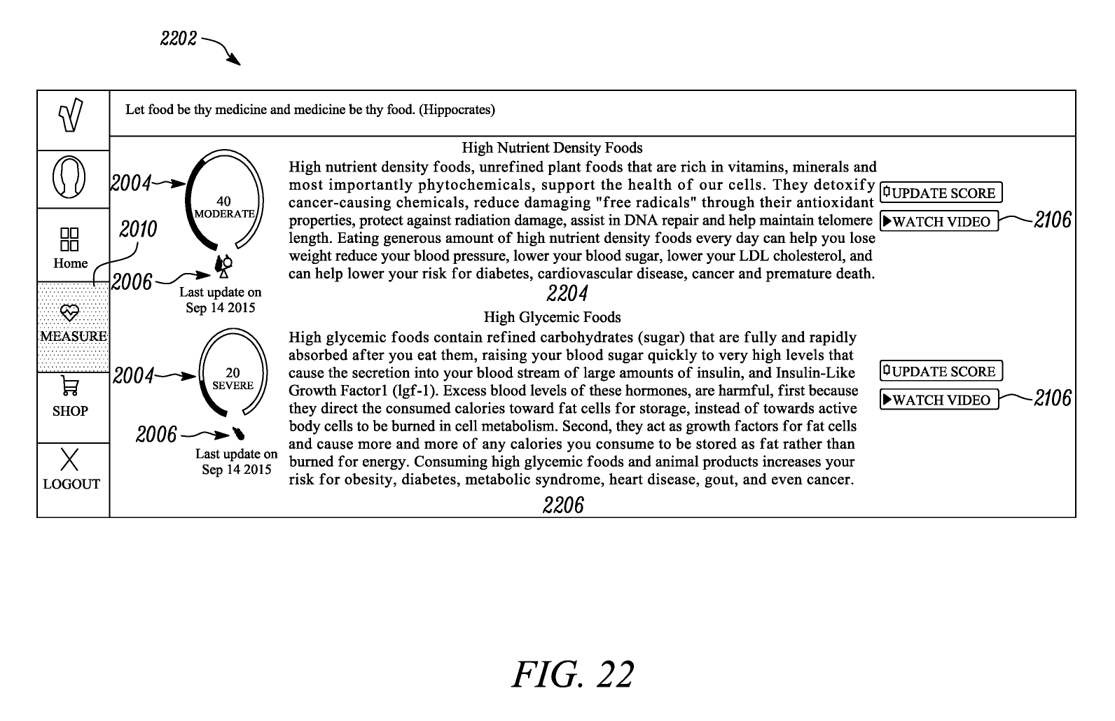

FIG. 22 illustrates an exemplary graphical user interface 2202 executed by the application 114 to display: data 2204 and 2206 (for example, text) characterizing respective explanations of the severities for the displayed biomarkers 2004, and a button 2106 which when selected by the user makes the application 114 display data (for example, video) including a treatment recommendation based on the severity 2008 of the particular biomarker 2006 corresponding to the selected button, according to some implementations of the current subject matter.

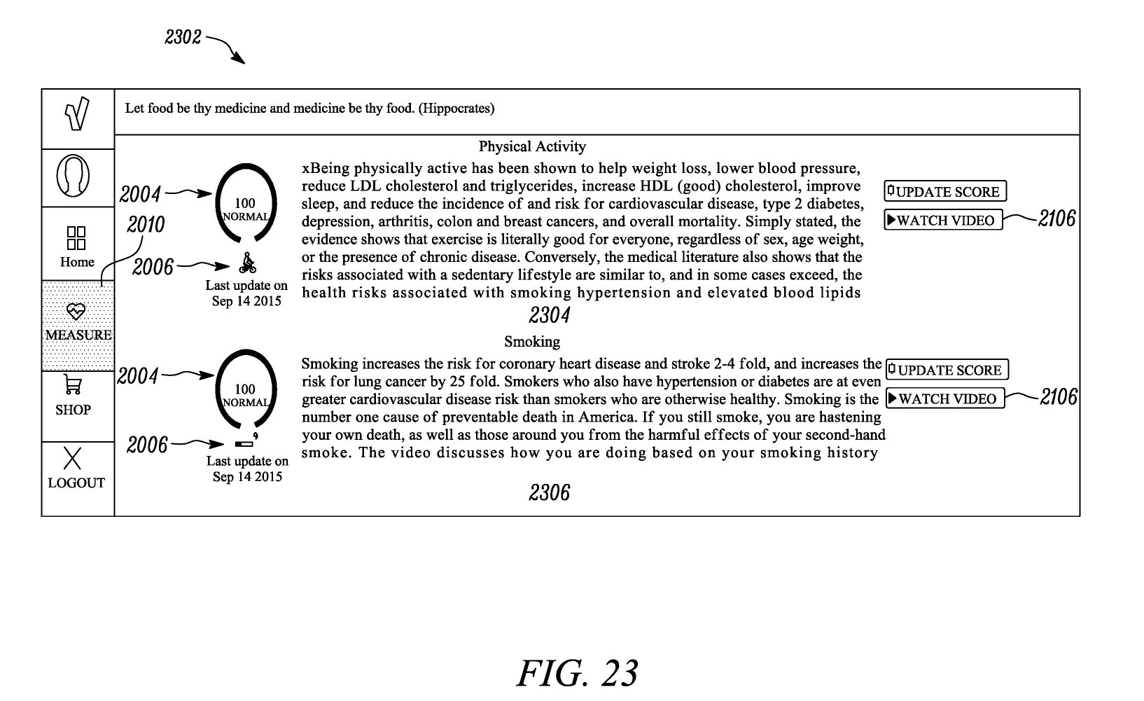

FIG. 23 illustrates an exemplary graphical user interface 2302 executed by the application 114 to display: data 2304 and 2306 (for example, text) characterizing respective explanations of the severities for the displayed biomarkers 2004, and a button 2106 which when selected by the user makes the application 114 display data (for example, video) including a treatment recommendation based on the severity 2008 of the particular biomarker 2006 corresponding to the selected button, according to some implementations of the current subject matter.

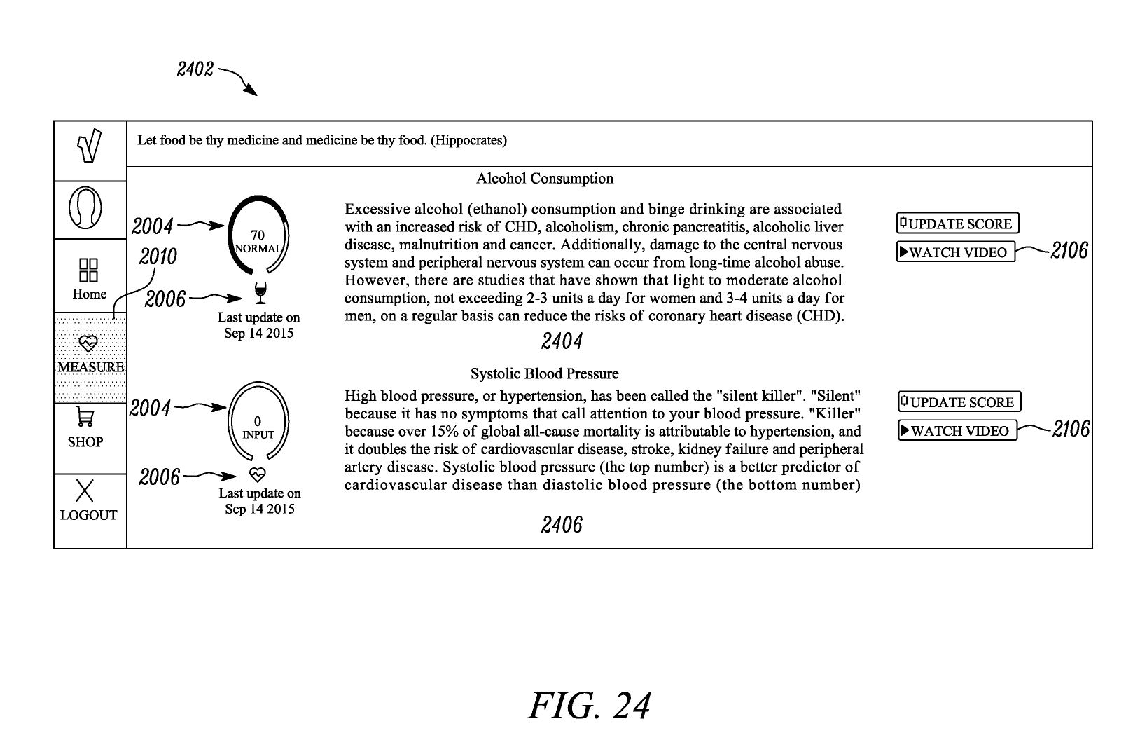

FIG. 24 illustrates an exemplary graphical user interface 2402 executed by the application 114 to display: data 2404 and 2406 (for example, text) characterizing respective explanations of the severities for the displayed biomarkers 2004, and a button 2106 which when selected by the user makes the application 114 display data (for example, video) including a treatment recommendation based on the severity 2008 of the particular biomarker 2006 corresponding to the selected button, according to some implementations of the current subject matter.

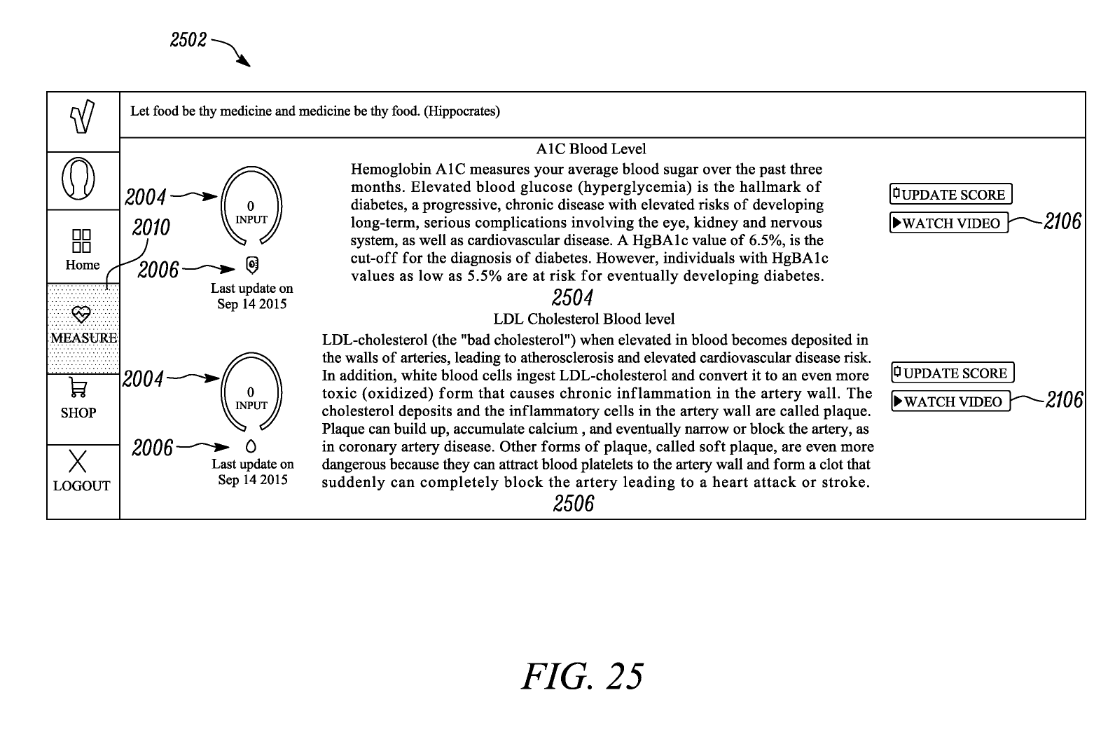

FIG. 25 illustrates an exemplary graphical user interface 2502 executed by the application 114 to display: data 2504 and 2506 (for example, text) characterizing respective explanations of the severities for the displayed biomarkers 2004, and a button 2106 which when selected by the user makes the application 114 display data (for example, video) including a treatment recommendation based on the severity 2008 of the particular biomarker 2006 corresponding to the selected button, according to some implementations of the current subject matter.

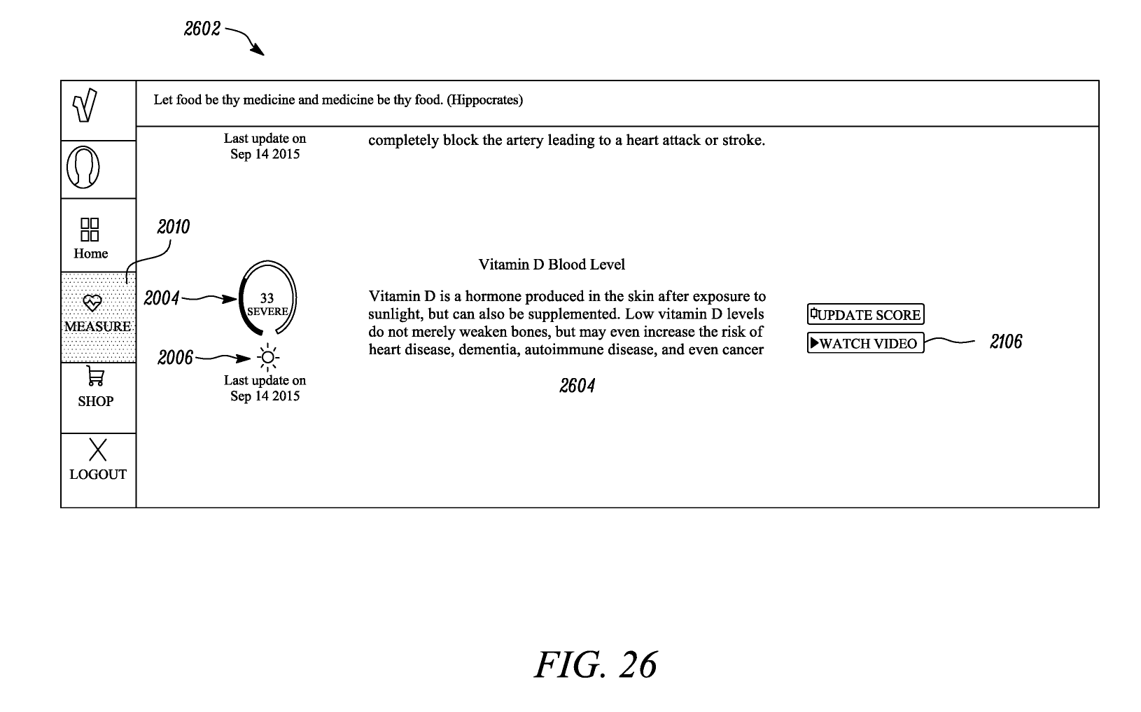

FIG. 26 illustrates an exemplary graphical user interface 2602 executed by the application 114 to display: data 2604 (for example, text) characterizing an explanation of the severity for the displayed biomarker 2006, and a button 2106 which when selected by the user makes the application 114 display data (for example, video) including a treatment recommendation based on the severity 2008 of that particular biomarker 2006, according to some implementations of the current subject matter.

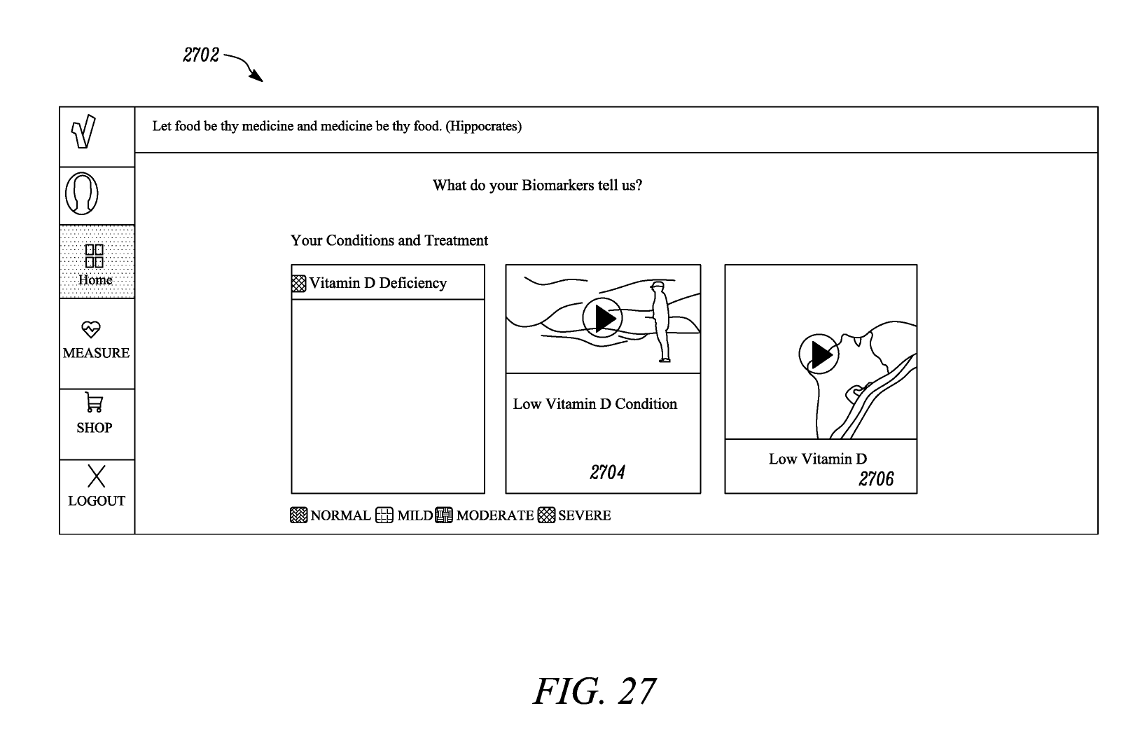

FIG. 27 illustrates an exemplary graphical user interface 2702 executed by the application 114 to display a link 2704 to data (for example, text such as 2604) characterizing an explanation of a physiological condition when the user has the severity determined by the assessment module 124, and another link 2706 (which is similar to button 2106) to data (for example, a video) of at least one treatment recommendation for the user, according to some implementations of the current subject matter.

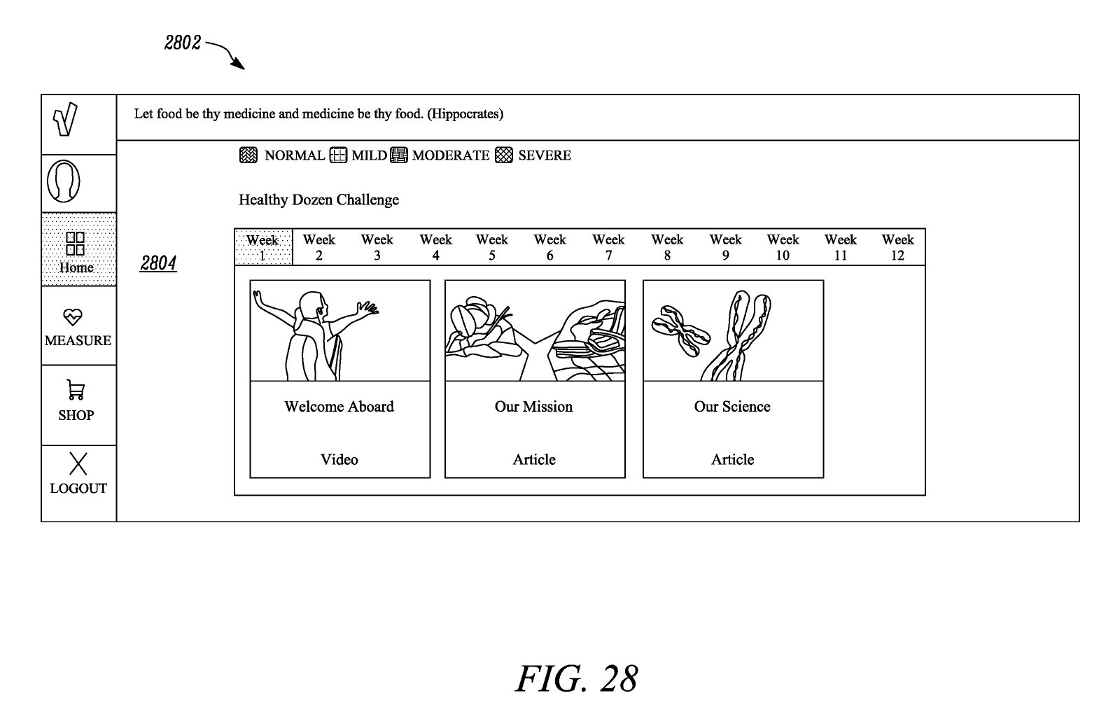

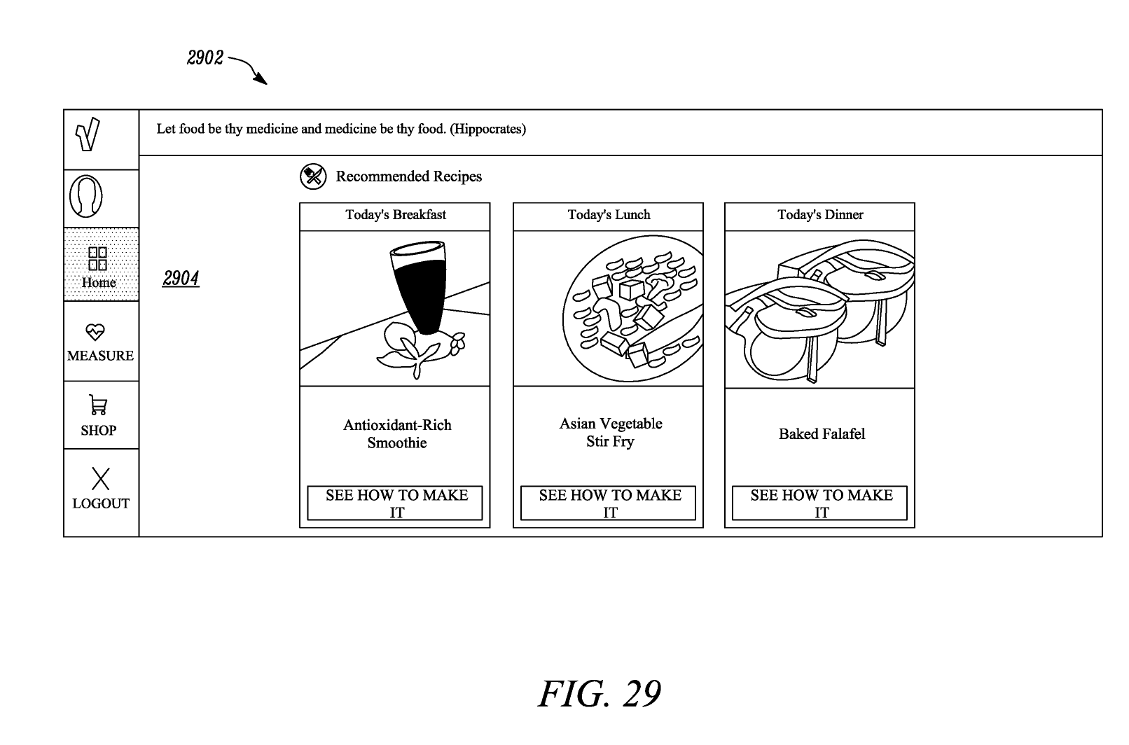

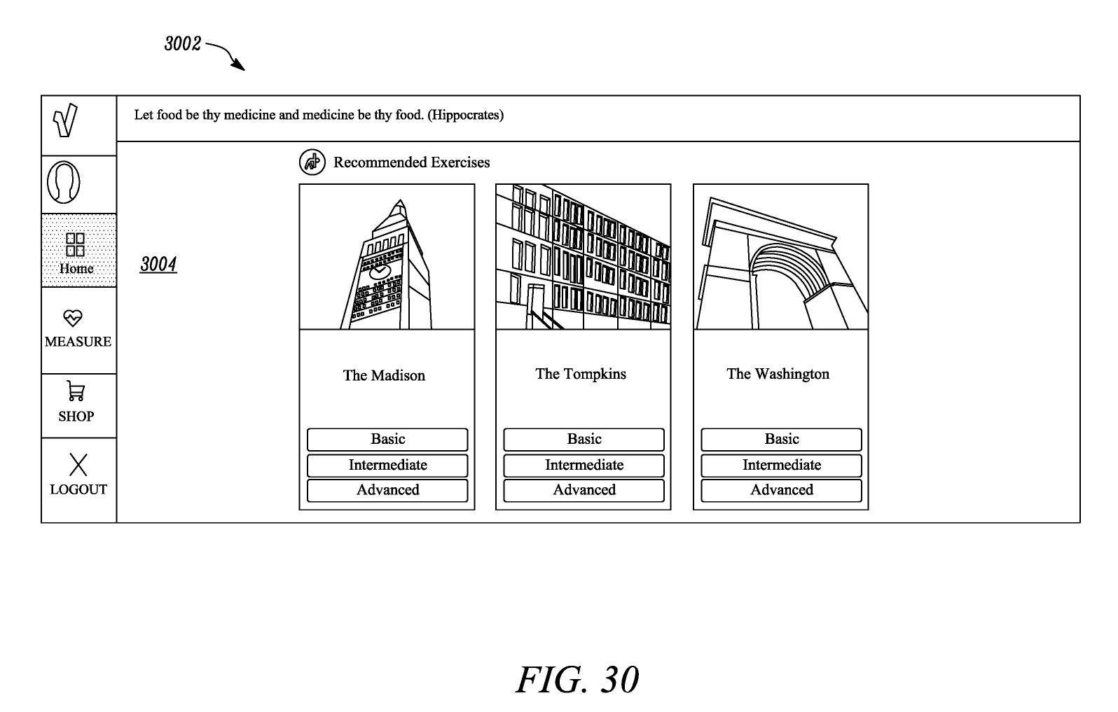

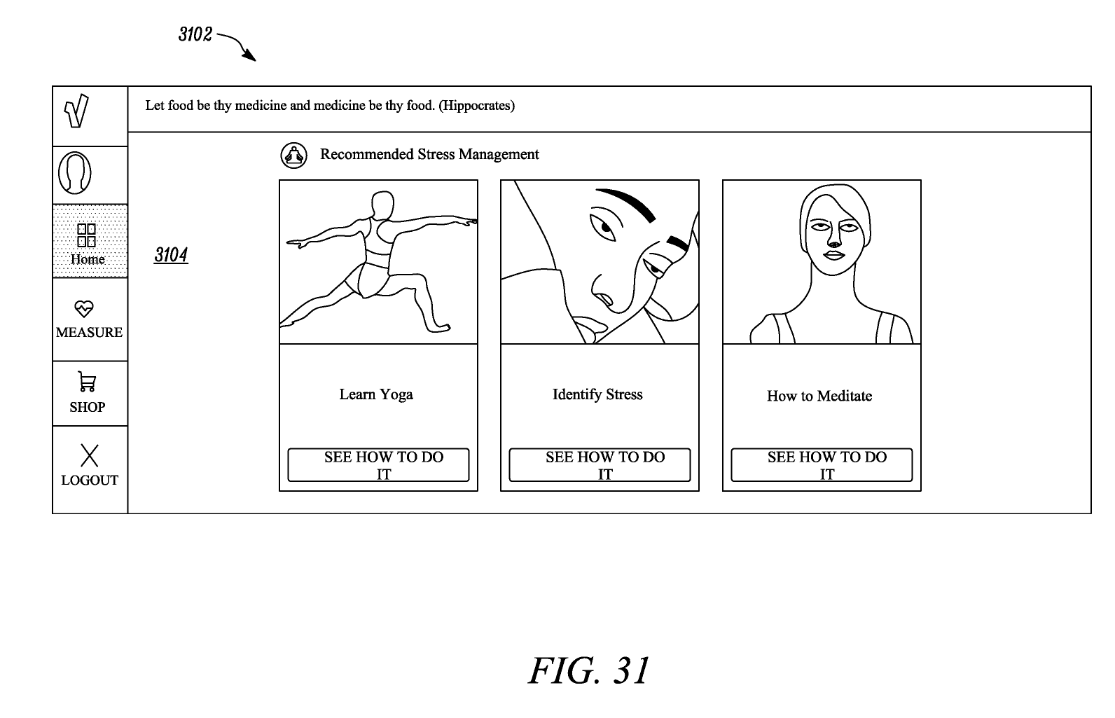

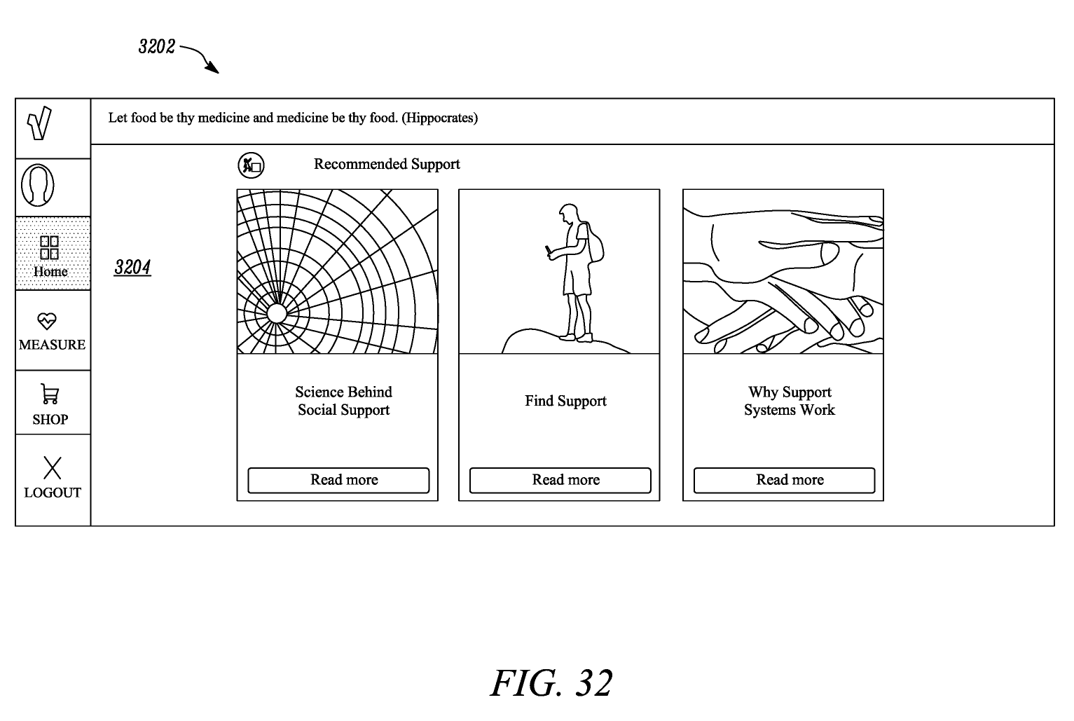

FIG. 28 illustrates an exemplary graphical user interface 2802 executed by the application 114 to display a challenge program 2804, which is one of multiple challenge programs that a user can select, according to some implementations of the current subject matter. Those multiple challenge programs can be stored in the database 130. The display module 120 can retrieve the content of the selected challenge program from the database 130 via the persistence module 128. The content of the challenge program can include recommended recipes (as discussed below by FIG. 29), recommended exercises (as discussed below by FIG. 30), recommended stress management (as discussed below by FIG. 31), and recommended support (as discussed below by FIG. 32). The display module 120 can then display the content of the selected challenge program on the application 114 at the computing device 110. In each challenge program, the application 114 can present preset challenges per week. The application 114 can display a request to the user to update the biomarker values after taking every week (or, in alternate implementations, after any other preset time period) of challenge. Once the user modifies the biomarker value for any biomarker, the assessment module can instantly recalculate the biomarker score for that biomarker, the severity for that biomarker, and the overall health score. The display module 120 can display the updated biomarker score for that biomarker, the severity for that biomarker, and the overall health score instantly.

FIG. 29 illustrates an exemplary graphical user interface 2902 executed by the application 114 to display recommended recipes 2904 within the challenge program 2804 shown in FIG. 28, according to some implementations of the current subject matter.

FIG. 30 illustrates an exemplary graphical user interface 3002 executed by the application 114 to display recommended exercises 3004 within the challenge program 2804 shown in FIG. 28, according to some implementations of the current subject matter.

FIG. 31 illustrates an exemplary graphical user interface 3102 executed by the application 114 to display recommended stress management activities 3104 within the challenge program 2804 shown in FIG. 28, according to some implementations of the current subject matter.

FIG. 32 illustrates an exemplary graphical user interface 3202 executed by the application 114 to display recommended human support activities 3204 within the challenge program 2804 shown in FIG. 28, according to some implementations of the current subject matter.

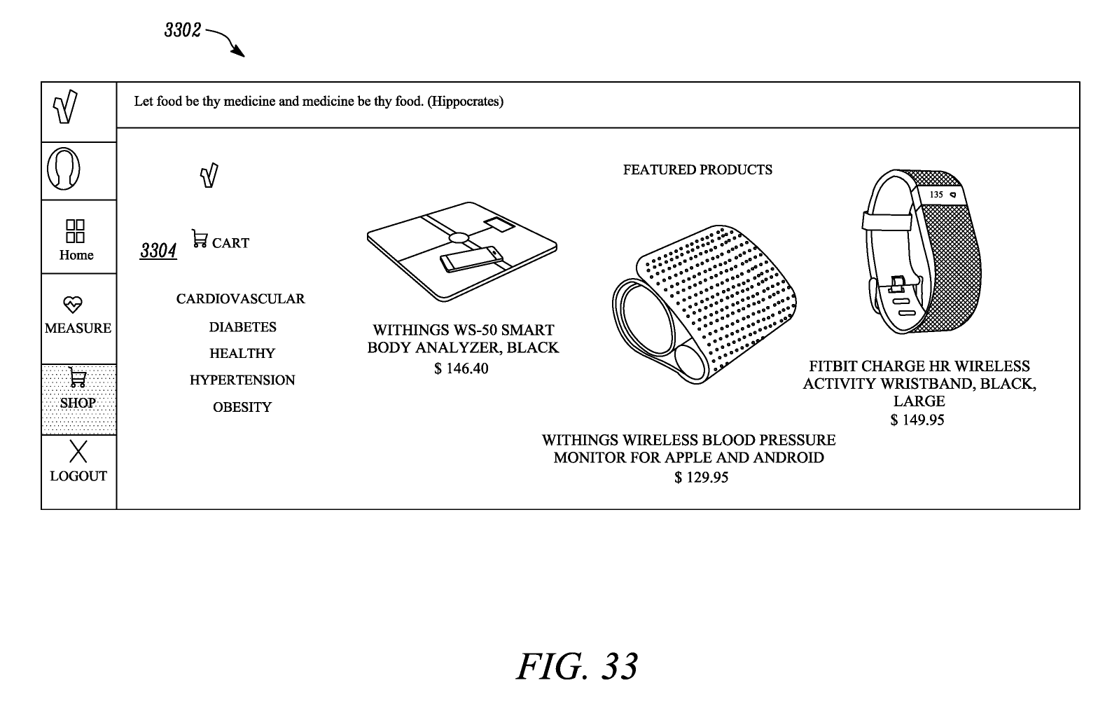

FIG. 33 illustrates an exemplary graphical user interface 3302 executed by the application 114 to display an electronic store 3304 that sells third party devices, such as wearable devices, from which the interaction module 118 can receive values of at least some biomarkers, according to one variation of the current subject matter.

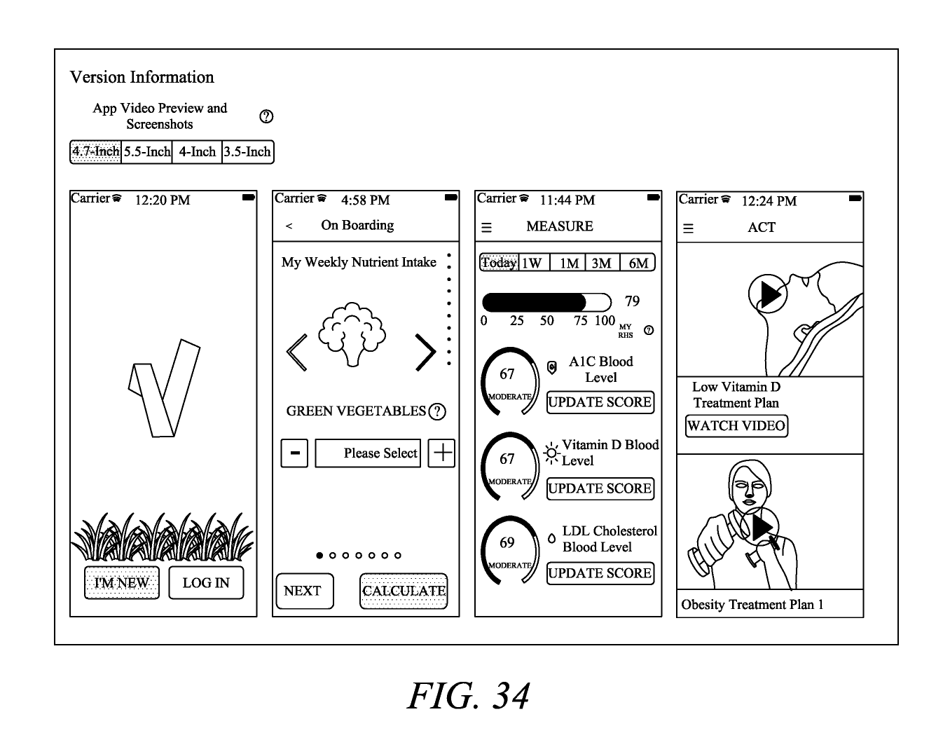

FIG. 34 illustrates exemplary display screens of the application 114 on some types of computing devices 110, such as a particular smartphone (for example, IPHONE, ANDROID based phone, and so on), according to some implementations of the current subject matter.

Note that in instances where the user inputs biomarker values of another individual, the term user used in the current subject matter can be appropriately changed to individual. For example, the system 102 in this case generates scores, severities and treatment recommendations for that individual rather than the user. Various implementations of the subject matter described herein can be realized/implemented in digital electronic circuitry, integrated circuitry, specially designed application specific integrated circuits (ASICs), computer hardware, firmware, software, and/or combinations thereof. These various implementations can be implemented in one or more computer programs. These computer programs can be executable and/or interpreted on a programmable system. The programmable system can include at least one programmable processor, which can be have a special purpose or a general purpose. The at least one programmable processor can be coupled to a storage system, at least one input device, and at least one output device. The at least one programmable processor can receive data and instructions from, and can transmit data and instructions to, the storage system, the at least one input device, and the at least one output device.

These computer programs (also known as programs, software, software applications or code) can include machine instructions for a programmable processor, and can be implemented in a high-level procedural and/or object-oriented programming language, and/or in assembly/machine language. As can be used herein, the term “machine-readable medium” can refer to any computer program product, apparatus and/or device (for example, magnetic discs, optical disks, memory, programmable logic devices (PLDs)) used to provide machine instructions and/or data to a programmable processor, including a machine-readable medium that can receive machine instructions as a machine-readable signal. The term “machine-readable signal” can refer to any signal used to provide machine instructions and/or data to a programmable processor.

To provide for interaction with a user, the subject matter described herein can be implemented on a computer that can display data to one or more users on a display device, such as a cathode ray tube (CRT) device, a liquid crystal display (LCD) monitor, a light emitting diode (LED) monitor, or any other display device. The computer can receive data from the one or more users via a keyboard, a mouse, a trackball, a joystick, or any other input device. To provide for interaction with the user, other devices can also be provided, such as devices operating based on user feedback, which can include sensory feedback, such as visual feedback, auditory feedback, tactile feedback, and any other feedback. The input from the user can be received in any form, such as acoustic input, speech input, tactile input, or any other input.

The subject matter described herein can be implemented in a computing system that can include at least one of a back-end component, a middleware component, a front-end component, and one or more combinations thereof. The back-end component can be a data server. The middleware component can be an application server. The front-end component can be a client computer having a graphical user interface or a web browser, through which a user can interact with an implementation of the subject matter described herein. The components of the system can be interconnected by any form or medium of digital data communication, such as a communication network. Examples of communication networks can include a local area network, a wide area network, internet, intranet, Bluetooth network, infrared network, or other networks.

The computing system can include clients and servers. A client and server can be generally remote from each other and can interact through a communication network. The relationship of client and server can arise by virtue of computer programs running on the respective computers and having a client-server relationship with each other.

Although a few variations have been described in detail above, other modifications can be possible. For example, the logic flows depicted in the accompanying figures and described herein do not require the particular order shown, or sequential order, to achieve desirable results. Other implementations may be within the scope of the claims.

As used herein, “reducing a likelihood of developing” a particular physiological condition or disease means to delay and/or postpone development of the physiological condition or disease. This delay can be of varying lengths of time, depending on the history of the disease and/or individual being treated. As is evident to one skilled in the art, a sufficient or significant delay can, in effect, encompass prevention, in that the individual does not develop the physiological condition or disease. A method that reduces a likelihood of developing one or more physiological conditions is a method that reduces the probability of disease development in a given time frame and/or reduces the extent of the physiological condition or disease or its complications in a given time frame, when compared to not using the method. Such comparisons are typically based on studies using a statistically significant number of subjects. “Developing” may also refer to disease progression that may be initially undetectable and includes occurrence, recurrence, and onset.

As used herein, the phrase “aiding in the reduction of a physiological condition” means any of decreasing or reducing one or more symptoms of a physiological condition (such as, a chronic disease), preventing an individual from developing a physiological condition (such as, an individual predisposed for developing a physiological condition, such as a chronic disease) and/or reducing the likelihood that an individual will develop a physiological condition (such as a chronic disease). In some implementations, the systems, methods, non-transitory computer programmable products, and/or articles described herein aid in the reduction of one or more physiological conditions such as, but not limited to, chronic diseases including cardiovascular disease, diabetes, hypertension, and/or obesity.

As used herein, the term “individual” or “subject” or “user” refers to a vertebrate, such as a mammal or a human. Mammals include, but are not limited to, murines, simians, humans, farm animals, sport animals, companion animals, and pets.

As used herein, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that a feature or element that is not recited is also permissible.

As used herein, the singular terms “a,” “an,” and “the” may include the plural reference unless the context clearly indicates otherwise.

A composition or method described herein as “comprising” or “including” one or more named elements or steps is open-ended, meaning that the named elements or steps are essential, but other elements or steps may be added within the scope of the composition or method. To avoid prolixity, it is also understood that any composition or method described as “comprising” or “including” (or “comprises” or “includes”) one or more named elements or steps also describes the corresponding, more limited, composition or method “consisting essentially of” (or “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and close-ended composition or method “consisting of” (or “consists of”) the named elements or steps to the exclusion of any other unnamed element or step. In any composition or method disclosed herein, known or disclosed equivalents of any named essential element or step may be substituted for that element or step.

Unless defined otherwise herein, all technical and scientific terms used herein have the same meaning as commonly understood by one of ordinary skill in the art to which the current subject matter pertains.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

What is claimed is:

1. A method comprising:
receiving, by one or more processors, values of a plurality of biomarkers from an application executed on a computing device of a user;
generating, by the one or more processors, a plurality of biomarker scores for the plurality of biomarkers;
computing, by the one or more processors, a severity associated with each biomarker of the plurality of biomarkers, the severity for the biomarker being computed based on a biomarker score of the plurality of the biomarker scores that is associated with the biomarker;
identifying, by the one or more processors, a physiological condition and at least one treatment recommendation that are specific to the severity of each biomarker;
retrieving, by the one or more processors and from a database operably coupled to the one or more processors, one or more links to an explanation of the physiological condition of each biomarker and at least one treatment recommendation for each biomarker; and
sending, by the one or more processors, the plurality of biomarker scores and the one or more links to the explanation of the severity and the at least one treatment recommendation to the application executed on the computing device of the user via a communication network.

2. The method of claim 1, further comprising:
computing, by the one or more processors, an overall heath score for a user by weighting and adding each biomarker score of the plurality of biomarker scores; and
sending, by the one or more processors, the overall health score to the application executed on the computing device of the user via a communication network.

3. The method of claim 2, wherein each biomarker is associated with a predetermined weight, the predetermined weight being used for weighting a biomarker score associated with the biomarker.

4. The method of claim 2, wherein the application simultaneously displays the plurality of biomarker scores, the one or more links to the explanation of the severity and the at least one treatment recommendation, and the overall health score on a single graphical user interface.

5. The method of claim 1, wherein the severity for each biomarker is one of: normal, mild, moderate and severe.

6. The method of claim 1, wherein the one or more processors are arranged in a model view controller (MVC) architecture.

7. The method of claim 1, wherein generating of the plurality of biomarker scores for the plurality of biomarkers comprises:
identifying a range within a plurality of ranges within which each value of the plurality of values lies; and
determining, using a table stored in the database, a biomarker score of the plurality of biomarker scores that is associated with the determined range.

8. The method of claim 1, wherein the retrieving of a link of the explanation of the physiological condition of each biomarker comprises identifying the explanation amongst a plurality of explanations for which separate links are stored in the database, the plurality of explanations comprising a separate explanation for each severity of each biomarker.

9. The method of claim 1, wherein the retrieving of a link of the at least one treatment recommendation for each biomarker comprises identifying the at least one treatment recommendation amongst a plurality of treatment recommendations for which separate links are stored in the database, the plurality of treatment recommendations comprising a separate treatment recommendation for each severity of each biomarker.

10. A non-transitory computer program product storing instructions that, when executed by at least one programmable processor, cause the at least one programmable processor to perform operations comprising:
receiving values of a plurality of biomarkers from an application executed on a computing device of a user;
generating a plurality of biomarker scores for the plurality of biomarkers;
computing an overall heath score for a user by weighting and adding each biomarker score of the plurality of biomarker scores; and
sending the overall health score to the application executed on the computing device of the user via a communication network.

11. The non-transitory computer program product of claim 10, wherein the operations further comprise:
computing a severity associated with each biomarker of the plurality of biomarkers, the severity for the biomarker being computed based on a biomarker score of the plurality of the biomarker scores that is associated with the biomarker;
identifying a physiological condition and at least one treatment recommendation that are specific to the severity of each biomarker;
retrieving, from a database operably coupled to the one or more processors, one or more links to an explanation of the physiological condition of each biomarker and at least one treatment recommendation for each biomarker; and
sending the plurality of biomarker scores and the one or more links to the explanation of the severity and the at least one treatment recommendation to the application executed on the computing device of the user via a communication network.

12. The non-transitory computer program product of claim 11, wherein the application simultaneously displays the plurality of biomarker scores, the one or more links to the explanation of the severity and the at least one treatment recommendation, and the overall health score on a single graphical user interface.

13. A system comprising:
a frontend component of a model view controller (MVC) architecture, the frontend component comprising an interaction module and a display module, the interaction module configured to receive a plurality of values of a plurality of biomarkers from a computing device; and
a backend component of the MVC architecture, the backend component comprising an assessment module and a recommendation module, the assessment module configured to generate a plurality of biomarker scores for the plurality of biomarkers based on the values of the biomarkers, the assessment module configured to determine a severity associated with each biomarker based on a biomarker score for the biomarker, the recommendation module configured to determine a treatment recommendation specific to each biomarker based on the severity for the biomarker, the assessment module configured to send the plurality of biomarker scores to the display module, the recommendation module configured to send each treatment recommendation to the display module.

14. The system of claim 13, wherein the computing device executes an application configured to receive one or more values of the plurality of values from the computing device, the interaction module configured to receive the one or more values from the application.

15. The system of claim 13, wherein the display module enables the display of the plurality of biomarker scores and the treatment recommendation specific to each biomarker on the computing device.

16. The system of claim 13, wherein:
the frontend component further comprises a login module configured to receive authentication data of a user from the computing device; and
the backend component further comprises a user module configured to receive the authentication data from the login module to authenticate the user, the assessment module generating the plurality of biomarker scores after the authentication of the user.

17. The system of claim 13, further comprising:
a database configured to store a plurality of treatment recommendations that include a treatment recommendation specific to each severity level of each biomarker.

18. The system of claim 17, wherein the backend component further comprises a persistence module, the persistence module being operably coupled to the database, the plurality of treatment recommendations stored in the database being accessible via the persistence module.
